SEQUENCE LISTING (142 sequences)

SEQ ID NO: 1 - protein, 34 aa, Artificial
  zinc finger
XXXCXXXXCXXXXXXXXXXXXHXXXXXXXHXXXX

SEQ ID NO: 2 - protein, 25 aa, Artificial
  first class of ZFPs
CXXXXCXXXXXXXXXXXXHXXXXXH

SEQ ID NO: 3 - protein, 4 aa, Artificial
  C3H ZFP
CCHC

SEQ ID NO: 4 - protein, 34 aa, Artificial
  zinc finger
XXXXXXXXCXXXXXXXXXXXXHXXXXXXXHXXXX

SEQ ID NO: 5 - protein, 34 aa, Artificial
  zinc finger
XXXCXXXXXXXXXXXXXXXXXHXXXXXXXHXXXX

SEQ ID NO: 6 - protein, 34 aa, Artificial
  zinc finger
XXXCXXXXCXXXXXXXXXXXXXXXXXXXXHXXXX

SEQ ID NO: 7 - protein, 34 aa, Artificial
  zinc finger
XXXCXXXXCXXXXXXXXXXXXHXXXXXXXXXXXX

SEQ ID NO: 8 - protein, 34 aa, Artificial
  zinc finger
XXXXXXXXXXXXXXXXXXXXXHXXXXXXXHXXXX

SEQ ID NO: 9 - protein, 34 aa, Artificial
  zinc finger
XXXXXXXXCXXXXXXXXXXXXXXXXXXXXHXXXX

SEQ ID NO: 10 - protein, 34 aa, Artificial
  zinc finger
XXXXXXXXCXXXXXXXXXXXXHXXXXXXXXXXXX

SEQ ID NO: 11 - protein, 34 aa, Artificial
  zinc finger
XXXCXXXXXXXXXXXXXXXXXXXXXXXXXHXXXX

SEQ ID NO: 12 - protein, 34 aa, Artificial
  zinc finger
XXXCXXXXXXXXXXXXXXXXXHXXXXXXXXXXXX

SEQ ID NO: 13 - protein, 34 aa, Artificial
  zinc finger
XXXCXXXXCXXXXXXXXXXXXXXXXXXXXXXXXX

SEQ ID NO: 14 - protein, 34 aa, Artificial
  zinc finger
XXXCXXXXXXXXXXXXXXXXXXXXXXXXXXXXXX

SEQ ID NO: 15 - protein, 34 aa, Artificial
  zinc finger
XXXXXXXXCXXXXXXXXXXXXXXXXXXXXXXXXX

SEQ ID NO: 16 - protein, 34 aa, Artificial
  zinc finger
XXXXXXXXXXXXXXXXXXXXXHXXXXXXXXXXXX

SEQ ID NO: 17 - protein, 34 aa, Artificial
  zinc finger
XXXXXXXXXXXXXXXXXXXXXXXXXXXXXHXXXX

SEQ ID NO: 18 - protein, 34 aa, Artificial
  zinc finger
XXXXXXXXXXXXXXXXXXXXXXXXXXXXXXXXXX

SEQ ID NO: 19 - protein, 5 aa, Artificial
  linker
TGEKP

SEQ ID NO: 20 - protein, 5 aa, Artificial
  linker
GGGGS

SEQ ID NO: 21 - protein, 8 aa, Artificial
  linker
GGRRGGGS

SEQ ID NO: 22 - protein, 9 aa, Artificial
  linker
LRQRDGERP

SEQ ID NO: 23 - protein, 12 aa, Artificial
  linker
LRQKDGGGSERP

SEQ ID NO: 24 - protein, 16 aa, Artificial
  linker
LRQKDGGGSGGGSERP

SEQ ID NO: 25 - protein, 28 aa, Artificial
  SP-1 consensus
YKCPECGKSFSXXXXXXXHQRTHTGEKP

SEQ ID NO: 26 - protein, 34 aa, Artificial
  F1
KKKSKGHECPICFRVFKXXXXXXXHKRSHTGEKP

SEQ ID NO: 27 - protein, 28 aa, Artificial
  F2
YKCTVCGKSFSXXXXXXXHKRLHTGEKP

SEQ ID NO: 28 - protein, 23 aa, Artificial
  F3
FSCNYCQRKFYXXXXXXXHVRIH

SEQ ID NO: 29 - protein, 4 aa, Artificial
  C-terminal sequence to the third finger of SP-1
QNKK

SEQ ID NO: 30 - protein, 5 aa, Artificial
  helix capping sequence
TGEXX

SEQ ID NO: 31 - protein, 32 aa, Artificial
  zinc finger backbone
KSKGHECPICFRVFKXXXXXXXHKRSHTGEKP

SEQ ID NO: 32 - protein, 28 aa, Artificial
  zinc finger backbone
YKCTVCGKSFSXXXXXXXHKRLHTGEKP

SEQ ID NO: 33 - protein, 27 aa, Artificial
  zinc finger backbone
FSCNYCQRKFGXXXXXXXHVRIHQNKK

SEQ ID NO: 34 - DNA, 60 bp, Artificial
  H1
ctcaccggtgtgagaacgcttgtgnnnnnnnnnnnnnnnnnnnnncttgaaaacacggaa

SEQ ID NO: 35 - DNA, 60 bp, Artificial
  H2
ttcaccagtatgaagacgcttatgnnnnnnnnnnnnnnnnnnnnnagaaaaagacttacc

SEQ ID NO: 36 - DNA, 63 bp, Artificial
  H3
cttcttgttctggtggatacgcacgtgnnnnnnnnnnnnnnnnnnnnnaccgaacttacgctg

SEQ ID NO: 37 - DNA, 44 bp, Artificial
  PB1
aagtctaagggtcacgagtgcccaatctgcttccgtgttttcaa

SEQ ID NO: 38 - DNA, 54 bp, Artificial
  PB2
tctcacaccggtgagaagccatacaagtgcactgtttgtggtaagtctttttct

SEQ ID NO: 39 - DNA, 54 bp, Artificial
  PB3
cttcatactggtgaaaagccattctcttgcaactactgccagcgtaagttcggt

SEQ ID NO: 40 - DNA, 10 bp, Artificial
  ZFP1 target
gtggacgagt

SEQ ID NO: 41 - protein, 7 aa, Artificial
  ZFP1 F1
RSDNLAR

SEQ ID NO: 42 - protein, 7 aa, Artificial
  ZFP1 F2
DRSNLTR

SEQ ID NO: 43 - protein, 7 aa, Artificial
  ZFP1 F3
RSDALTR

SEQ ID NO: 44 - DNA, 10 bp, Artificial
  ZFP 2 target
cgggatgggt

SEQ ID NO: 45 - protein, 7 aa, Artificial
  ZFP2 F1
RSDHLAR

SEQ ID NO: 46 - protein, 7 aa, Artificial
  ZFP2 F2
TSGNLVR

SEQ ID NO: 47 - protein, 7 aa, Artificial
  ZFP2 F3
RSDHLTE

SEQ ID NO: 48 - DNA, 10 bp, Artificial
  ZFP3 target
tggtgggtgt

SEQ ID NO: 49 - protein, 7 aa, Artificial
  ZFP3 F1
RSDALTR

SEQ ID NO: 50 - protein, 7 aa, Artificial
  ZFP3 F2
RSDHLTT

SEQ ID NO: 51 - protein, 7 aa, Artificial
  ZFP3 F3
RSDHLTT

SEQ ID NO: 52 - DNA, 10 bp, Artificial
  ZFP4 target
gaagaggatt

SEQ ID NO: 53 - protein, 7 aa, Artificial
  ZFP4 F1
QSSNLAR

SEQ ID NO: 54 - protein, 7 aa, Artificial
  ZFP4 F2
RSDNLAR

SEQ ID NO: 55 - protein, 7 aa, Artificial
  ZFP4 F3
QSGNLTR

SEQ ID NO: 56 - DNA, 10 bp, Artificial
  ZFP5 target
gaggaagggg

SEQ ID NO: 57 - protein, 7 aa, Artificial
  ZFP5 F1
RSDHLAR

SEQ ID NO: 58 - protein, 7 aa, Artificial
  ZFP5 F2
QSGNLAR

SEQ ID NO: 59 - protein, 7 aa, Artificial
  ZFP5 F3
RSDNLTR

SEQ ID NO: 60 - DNA, 9 bp, Artificial
  ZFP6 target
tgggtagtc

SEQ ID NO: 61 - protein, 7 aa, Artificial
  ZFP6 F1
ERGTLAR

SEQ ID NO: 62 - protein, 7 aa, Artificial
  ZFP6 F2
QSGSLTR

SEQ ID NO: 63 - protein, 7 aa, Artificial
  ZFP6 F3
RSDHLTT

SEQ ID NO: 64 - DNA, 10 bp, Artificial
  ZFP7 target
ggggaaaggg

SEQ ID NO: 65 - protein, 7 aa, Artificial
  ZFP7 F1
RSDHLTQ

SEQ ID NO: 66 - protein, 7 aa, Artificial
  ZFP7 F2
QSGNLAR

SEQ ID NO: 67 - protein, 7 aa, Artificial
  ZFP7 F3
RSDHLSR

SEQ ID NO: 68 - DNA, 10 bp, Artificial
  ZFP8 target
gaagagggtg

SEQ ID NO: 69 - protein, 7 aa, Artificial
  ZFP8 F1
QSSHLAR

SEQ ID NO: 70 - protein, 7 aa, Artificial
  ZFP8 F2
RSDNLAR

SEQ ID NO: 71 - protein, 7 aa, Artificial
  ZFP8 F3
QSGNLAR

SEQ ID NO: 72 - DNA, 10 bp, Artificial
  ZFP9 target
gaggaggatg

SEQ ID NO: 73 - protein, 7 aa, Artificial
  ZFP9 F1
QSSNLQR

SEQ ID NO: 74 - protein, 7 aa, Artificial
  ZFP9 F2
RSDNALR

SEQ ID NO: 75 - protein, 7 aa, Artificial
  ZFP9 F3
RSDNLQR

SEQ ID NO: 76 - DNA, 10 bp, Artificial
  ZFP10 target
gaggaggagg

SEQ ID NO: 77 - protein, 7 aa, Artificial
  ZFP10 F1
RSDNALR

SEQ ID NO: 78 - protein, 7 aa, Artificial
  ZFP10 F2
RSDNLAR

SEQ ID NO: 79 - protein, 7 aa, Artificial
  ZFP10 F3
RSDNLTR

SEQ ID NO: 80 - DNA, 10 bp, Artificial
  ZFP11 target
gtggcggctg

SEQ ID NO: 81 - protein, 7 aa, Artificial
  ZFP11 F1
QSSDLRR

SEQ ID NO: 82 - protein, 7 aa, Artificial
  ZFP11 F2
RSDELQR

SEQ ID NO: 83 - protein, 7 aa, Artificial
  ZFP11 F3
RSDALTR

SEQ ID NO: 84 - DNA, 9 bp, Artificial
  ZFP12 target
tggggagat

SEQ ID NO: 85 - protein, 7 aa, Artificial
  ZFP12 F1
QSSNLAR

SEQ ID NO: 86 - protein, 7 aa, Artificial
  ZFP12 F2
QSGHLQR

SEQ ID NO: 87 - protein, 7 aa, Artificial
  ZFP12 F3
RSDHLTT

SEQ ID NO: 88 - DNA, 9 bp, Artificial
  ZFP13 target
gaggaagct

SEQ ID NO: 89 - protein, 7 aa, Artificial
  ZFP13 F1
QSSDLRR

SEQ ID NO: 90 - protein, 7 aa, Artificial
  ZFP13 F2
QSGNLAR

SEQ ID NO: 91 - protein, 7 aa, Artificial
  ZFP13 F3
RSDNLTR

SEQ ID NO: 92 - DNA, 10 bp, Artificial
  ZFP14 target
gcttgtggct

SEQ ID NO: 93 - protein, 7 aa, Artificial
  ZFP14 F1
DRSHLTR

SEQ ID NO: 94 - protein, 7 aa, Artificial
  ZFP14 F2
TSGHLTT

SEQ ID NO: 95 - protein, 7 aa, Artificial
  ZFP14 F3
QSSDLTR

SEQ ID NO: 96 - DNA, 10 bp, Artificial
  ZFP15 target
gtagtggatg

SEQ ID NO: 97 - protein, 7 aa, Artificial
  ZFP15 F1
QSSNLAR

SEQ ID NO: 98 - protein, 7 aa, Artificial
  ZFP15 F2
RSDALSR

SEQ ID NO: 99 - protein, 7 aa, Artificial
  ZFP15 F3
QSGSLTR

SEQ ID NO: 100 - DNA, 10 bp, Artificial
  ZFP16 target
gtgtgggatt

SEQ ID NO: 101 - protein, 7 aa, Artificial
  ZFP16 F1
QSSNLAR

SEQ ID NO: 102 - protein, 7 aa, Artificial
  ZFP16 F2
RSDHLTT

SEQ ID NO: 103 - protein, 7 aa, Artificial
  ZFP16 F3
RSDALTR

SEQ ID NO: 104 - DNA, 18 bp, Artificial
  GMT forward primer
aatgatctcgcggctgct

SEQ ID NO: 105 - DNA, 20 bp, Artificial
  GMT reverse primer
gaatggctgatccaacgcat

SEQ ID NO: 106 - DNA, 29 bp, Artificial
  GMT probe
tcactcgctcataaggcttccttccaagt

SEQ ID NO: 107 - DNA, 21 bp, Artificial
  18S forward primer
tgcaacaaaccccgacttatg

SEQ ID NO: 108 - DNA, 19 bp, Artificial
  18S reverse primer
cccgcgtcgaccttttatc

SEQ ID NO: 109 - DNA, 16 bp, Artificial
  18S probe
aataaatgcgtccctt

SEQ ID NO: 110 - protein, 7 aa, Artificial
  ZFP C3 F1
QSSHLAR

SEQ ID NO: 111 - protein, 7 aa, Artificial
  ZFP C3 F2
QSSDLTR

SEQ ID NO: 112 - protein, 7 aa, Artificial
  ZFP C3 F3
TSGNLTR

SEQ ID NO: 113 - protein, 7 aa, Artificial
  ZFP C4 F1
QSSNLAR

SEQ ID NO: 114 - protein, 7 aa, Artificial
  ZFP C4 F2
QSGNLAR

SEQ ID NO: 115 - protein, 7 aa, Artificial
  ZFP C4 F3
RSDNLTR

SEQ ID NO: 116 - protein, 7 aa, Artificial
  ZFP C5 F1
RSDNLAR

SEQ ID NO: 117 - protein, 7 aa, Artificial
  ZFP C5 F2
QSGNLAR

SEQ ID NO: 118 - protein, 7 aa, Artificial
  ZFP C5 F3
QSGNLAR

SEQ ID NO: 119 - protein, 7 aa, Artificial
  ZFP C6 F1
QSGHLAR

SEQ ID NO: 120 - protein, 7 aa, Artificial
  ZFP C6 F2
TSGALTR

SEQ ID NO: 121 - protein, 7 aa, Artificial
  ZFP C6 F3
RSDNLTR

SEQ ID NO: 122 - protein, 7 aa, Artificial
  ZFP C7 F1
QSSNLAR

SEQ ID NO: 123 - protein, 7 aa, Artificial
  ZFP C7 F2
TSGNLTR

SEQ ID NO: 124 - protein, 7 aa, Artificial
  ZFP C7 F3
TSGNLTR

SEQ ID NO: 125 - protein, 7 aa, Artificial
  ZFP C8 F1
RSSNLAR

SEQ ID NO: 126 - protein, 7 aa, Artificial
  ZFP C8 F2
QSGHLQR

SEQ ID NO: 127 - protein, 7 aa, Artificial
  ZFP C8 F3
RSDHLRE

SEQ ID NO: 128 - protein, 7 aa, Artificial
  ZFP C9 F1
QSGNLAR

SEQ ID NO: 129 - protein, 7 aa, Artificial
  ZFP C9 F2
RSDALTT

SEQ ID NO: 130 - protein, 7 aa, Artificial
  ZFP C9 F3
RSDNLTT

SEQ ID NO: 131 - protein, 7 aa, Artificial
  ZFP C10 F1
DRSNLTR

SEQ ID NO: 132 - protein, 7 aa, Artificial
  ZFP C10 F2
RSDNLAR

SEQ ID NO: 133 - protein, 7 aa, Artificial
  ZFP C10 F3
QSGSLTR

SEQ ID NO: 134 - protein, 7 aa, Artificial
  ZFP C12 F1
DRSHLTR

SEQ ID NO: 135 - protein, 7 aa, Artificial
  ZFP C12 F2
TSGALTR

SEQ ID NO: 136 - protein, 7 aa, Artificial
  ZFP C12 F3
RSDNLTR

SEQ ID NO: 137 - DNA, 22 bp, Artificial
  cGMT forward primer
caatggaaagcggtgagcatat

SEQ ID NO: 138 - DNA, 18 bp, Artificial
  cGMT reverse primer
tccttcctcctggagccg

SEQ ID NO: 139 - DNA, 29 bp, Artificial
  cGMT probe
ctgacaaggccaagttcgtgaaggaattg

SEQ ID NO: 140 - DNA, 23 bp, Artificial
  GAPDH forward primer
gatcatcaagattgtatctgatc

SEQ ID NO: 141 - DNA, 22 bp, Artificial
  GAPDH reverse primer
cggttccttcgataactaagtc

SEQ ID NO: 142 - DNA, 22 bp, Artificial
  GAPDH probe
cggttccttcgataactaagtc

(19) United States
(12) Patent Application Publication
(10) Pub. No.: US 2004/0091990 A1
(43) Pub. Date: May 13, 2004

(54) Compositions and methods for regulation of plant gamma-tocopherol methyltransferase

(76) Inventors: Guofu Li, Johnston, IA (US); Qiang Liu, Foster City, CA (US); Andrew Jamieson, San Francisco, CA (US); Edward Rebar, El Cerrito, CA (US)

Correspondence Address: ROBINS & PASTERNAK, 1731 EMBARCADERO ROAD, SUITE 230, PALO ALTO, CA, 94303, US

(21) Appl. No.: 10/650,454

(22) Filed: Aug. 27, 2003

Related U.S. Application Data
(60) Provisional application No. 60/406,849, filed on Aug. 29, 2002.

(51) Int. Cl. C07H 21/04; C12N 9/10; C12N 15/82

(52) U.S. Cl. 435/193; 435/468; 536/23.2

(57) ABSTRACT

Disclosed herein are zinc finger proteins that bind to target sites in a plant gamma-tocopherol methyl transferase (GMT) gene; compositions comprising these GMT-targeted zinc finger proteins and methods of making and using such zinc finger proteins.

### CROSS-REFERENCE TO RELATED APPLICATIONS

This application claims the benefit of U.S. Provisional Application No. 60/406,849, filed Aug. 29, 2002, which application is hereby incorporated by reference in its entirety.

### TECHNICAL FIELD

The methods and compositions disclosed herein relate generally to the field of regulation of gene expression and specifically to methods of modulating expression of plant gamma-tocopherol methyltransferase (GMT) expression utilizing polypeptides derived from plant zinc finger-nucleotide binding proteins.

### BACKGROUND

Vitamin E (tocopherol) is a fat-soluble vitamin found in many vegetable seed oils, and leafy green vegetables. Vitamin E has many functions including acting as an antioxidant of lipids, protecting of cell membranes and prevention of damage to membrane associated enzymes. Vitamin E is present as alpha, beta and gamma tocopherols, with alpha-tocopherol forms having the most activity. Supplements may contain the alpha tocopherol that is either in the “d” form or a combination of the “d” and “l” forms. The “d” form is more active than the “l” form but when comparing supplements, an equivalent number of international units (IU) indicate equivalent activity. Less information is available about the action of the beta and gamma tocopherols, but they appear to have different antioxidant effects. Vitamin E supplementation in humans may have a variety of beneficial effects including slowing the progression of Alzheimer's disease, preventing heart disease, improving immune function in the elderly, reducing the risk of cataracts and decreasing the pain associated with arthritis.

Photosynthetic bacteria and higher plants share a common set of enzymatic reactions for tocopherol synthesis, in which gamma-tocopherol methyltransferase (GMT) catalyzes the conversion of gamma-tocopherol to alpha-tocopherol in the final step of vitamin E synthesis. The gene encoding GMT has been isolated and characterized from a variety of plant species including, pepper, soybean, Euglena, spinach and Arabidopsis. See, e.g., Shigeoka et al. (1992) Biochim Biophys Acta. 1128(2-3):220-6; GenBank Accession Nos. BM890961, AF213481 and AF104220). In many plant oils (the main dietary source of tocopherols), alpha-tocopherol is typically present in small amounts while high levels of its biosynthetic precursor, gamma-tocopherol are generally present. Attempts to overexpress GMT in order to produce crops with higher vitamin E content have shown that GMT overexpression can result in higher vitamin E levels. See, e.g., Shintani et al. (1998) Science 282:2098-2100. However, such attempts have been hampered in view of the lack of efficient and stable methods of gene regulation in a variety of crops and plants.

Thus, there remains a need compositions and methods for targeted regulation of the gamma-tocopherol methyltransferase (GMT) gene in plants to facilitate numerous applications such as, for example, the optimization of crop traits affecting nutritional value. In addition, such targeted regulation of GMT could be used to study biosynthetic pathways and gene function in plants.

### SUMMARY

In one aspect, the disclosure relates to a zinc finger protein that binds to a target site in a plant gamma-tocopherol methyl transferase (GMT) gene. Also disclosed is a zinc finger protein that modulates expression of a plant GMT as well as a zinc finger protein that, when present in plant cell, increases the amounts of vitamin E in the plant cell. Any of the zinc finger proteins described herein can be, for example, engineered (e.g., designed, selected and/or rearranged) and/or tandem arrays of plant sequences. Furthermore, the plant can be either a dicotyledenous plant (e.g., Brassica or Arabidopsis) or a monocotyledenous plant. Furthermore, any of the zinc finger proteins described herein can three component fingers, for example as shown in Table 1 and Table 3.

In another aspect, the disclosure relates to fusion polypeptides comprising any of the zinc finger proteins disclosed herein and at least one regulatory domain, for example an activation domain such as VP16.

In yet another aspect, the disclosure relates to isolated polynucleotides encoding any of the zinc finger proteins described herein. Expression vectors comprising these isolated polynucleotides are also described including, for example, expression vectors comprising plant promoters such as tissue-specific (e.g., seed- and/or leaf-specific) plant promoters.

In still further aspects, plant cells comprising any of the zinc finger proteins, isolated polynucleotides and/or expression vectors described herein are also provided.

In still further aspects, a transgenic plant comprising any of the isolated polynucleotides and/or expression vectors described herein are provided.

In yet another aspect, the disclosure relates to methods for modulating expression of GMT in a plant cell, for example by contacting the cell with any of the zinc finger proteins; isolated polynucleotides or expression vectors as described herein.

In another aspect, the disclosure relates to methods for increasing the amounts of vitamin E present in a plant cell by contacting the cell with any of the zinc finger proteins; isolated polynucleotides or expression vectors as described herein.

These and other embodiments will readily occur to those of skill in the art in light of the disclosure herein.

### BRIEF DESCRIPTION OF THE DRAWINGS

FIG. 1 is a schematic depicting construction of the YCF3 expression vector useful in expressing GMT-targeted plant ZFPs.

FIG. 2 shows the results of analysis of GMT mRNA in RNA isolated from Arabidopsis thaliana protoplasts transfected with constructs encoding fusion of a transcriptional activation domain with various Arabidopsis GMT-targeted plant ZFPs. Results are expressed as GMT mRNA normalized to 18S rRNA. AGMT numbers on the abscissa refer to the GMT-targeted plant ZFP binding domains shown in Table 1. Duplicate TaqMan® analyses are shown for each RNA sample.

FIG. 3 shows the results of analysis of activation of GMT in Brassica protoplasts transfected with constructs encoding fusion of a transcriptional activation domain with various canola GMT-targeted plant ZFPs. RNA was isolated from Brassica protoplasts and results are expressed as fold activation of GMT mRNA as normalized to GAPDH RNA. Designations on the abscissa refer to the GMT-targeted plant ZFP binding domains shown in Table 3. C1 refers to an activation domain only.

FIG. 4 shows the results of analysis of GMT mRNA in RNA isolated from transgenic Arabidopsis thaliana stably transformed with constructs encoding fusion of a transcriptional activation domain with an Arabidopsis GMT-targeted plant ZFP (AGMT-7). Results from individual plants are expressed as levels of GMT mRNA normalized to 18S rRNA. AGMT numbers on the abscissa refer to the GMT-targeted plant ZFP binding domains shown in Table 1 and include both canonical (C2H2) and non-canonical (C3H) recognition helices in a plant backbone. The average of duplicate TaqMan® analyses is shown for each RNA sample.

FIG. 5 shows the results of analysis of GMT mRNA in RNA isolated from transgenic Arabidopsis thaliana stably transformed with constructs encoding fusion of a transcriptional activation domain with an Arabidopsis GMT-targeted plant ZFP (AGMT-8). Results from individual plants are expressed as levels of GMT mRNA normalized to 18S rRNA. AGMT numbers on the abscissa refer to the GMT-targeted plant ZFP binding domains shown in Table 1 and include both canonical (C2H2) and non-canonical (C3H) recognition helices in a plant backbone. The average of duplicate TaqMan® analyses is shown for each RNA sample.

### DETAILED DESCRIPTION

### General

The present disclosure provides ZFPs that bind to target sites in plant gammatocopherol methyltransferase (GMT) genes, for example Arabidopsis and Brassica GMT genes. Also provided are methods of using these ZFPs along with host cells and transgenic plants comprising these ZFPs. The GMT-targeted ZFP can be a fusion polypeptide and, either by itself or as part of such a fusion, can enhance or suppress expression of GMT (i.e., modulate GMT gene expression). Polynucleotides encoding these ZFPs, and polynucleotides encoding fusion proteins comprising one or more of these ZFPs are also provided. Additionally provided are compositions comprising, in combination with an acceptable carrier, any of the zinc finger binding polypeptides described herein or functional fragments thereof; and compositions comprising a nucleotide sequence that encodes a GMT-binding zinc finger binding polypeptide or functional fragment thereof, wherein the GMT-targeted zinc finger polypeptide or functional fragment thereof binds to a cellular nucleotide sequence to modulate the function of GMT. Also provided are plant cells and transgenic plants comprising the GMT-targeted ZFPs (or polynucleotide encoding these ZFPs).

In additional embodiments, methods for modulating expression of GMT in plant cells, using ZFPs described herein are provided. For example, a GMT-targeted ZFP as described herein can be fused to an activation domain such that GMT is overexpressed (as compared to a control cell not containing the GMT-targeted ZFP). GMT overexpression results in increased vitamin E (tocopherol) production by the plant or plant cell. Thus, the methods and compositions described herein allow for the production of plant cells and whole plants in which the amount (or concentration) of Vitamin E in the cells or plants is increased as compared to a negative control. It will be clear to those of skill in the art that increased Vitamin E levels can also result from modulation of expression of genes other than GMT. For example, up-regulation of any gene in the pathway leading to alpha-tocopherol synthesis will result in increased Vitamin E levels in plants in which the gene is up-regulated.

The practice of the disclosed methods employs, unless otherwise indicated, conventional techniques in molecular biology, biochemistry, genetics, computational chemistry, cell culture, recombinant DNA and related fields as are within the skill of the art. These techniques are fully explained in the literature. See, for example, Sambrook et al. MOLECULAR CLONING: A LABORATORY MANUAL, Third Edition, Cold Spring Harbor Laboratory Press, 2001; Ausubel et al., CURRENT PROTOCOLS IN MOLECULAR BIOLOGY, John Wiley & Sons, New York, 1987 and periodic updates; and the series METHODS IN ENZYMOLOGY, Academic Press, San Diego.

The disclosures of all patents, patent applications and publications mentioned herein are hereby incorporated by reference in their entireties.

### Definitions

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally occurring amino acid, for example selenocysteine (Bock et al. (1991) Trends Biochem. Sci. 16:463-467; Nasim et al. (2000) J. Biol. Chem. 275:14,846-14,852) and the like.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity. A “binding profile” refers to a plurality of target sequences that are recognized and bound by a particular binding protein. For example, a binding profile can be determined by contacting a binding protein with a population of randomized target sequences to identify a sub-population of target sequences bound by that particular binding protein.

A “zinc finger binding protein” is a protein or segment within a larger protein that binds DNA, RNA and/or protein in a sequence-specific manner as a result of stabilization of protein structure through coordination of a zinc ion. The term zinc finger binding protein is often abbreviated as zinc finger protein or ZFP. A “canonical” zinc finger refers to a zinc-coordinating component (e.g., zinc finger) of a zinc finger protein having the general amino acid sequence: X₃-Cys-X₂₋₄-Cys-X₁₋₂-His-X₁₋₇-His-X₄ (SEQ ID NO:1) where X is any amino acid (also known as a C2H2 zinc finger). A “non-canonical” zinc finger refers to any type of finger other than a C2H2 zinc finger. Examples of non-canonical zinc fingers are described in U.S. patent application, Ser. No. Unassigned, filed Jan. 22, 2002, titled “Modified Zinc Finger Binding Proteins.”

A “designed” zinc finger protein is a protein not occurring in nature whose structure and composition results principally from rational criteria. Criteria for rational design include application of substitution rules and computerized algorithms for processing information in a database storing information of existing ZFP designs and binding data, for example as described in co-owned PCT WO 00/42219. A “selected” zinc finger protein is a protein not found in nature whose production results primarily from an empirical process such as phage display, two-hybrid systems and/or interaction trap assays. See e.g., U.S. Pat. No. 5,789,538; U.S. Pat. No. 6,007,988; U.S. Pat. No. 6,013,453; WO 95/19431; WO 96/06166; WO 98/54311 and Joung et al. (2000) Proc. Natl. Acad. Sci. USA 97:7382-7387. Selection methods also include ribosome display systems (e.g., PCT WO 00/27878) and mRNA-peptide fusion systems (e.g., U.S. Pat. No. 6,207,446; PCT WO 00/47775). Amino acid sequences of polypeptides (e.g., zinc fingers) obtained by selection or design are referred to as “adapted” amino acid sequences. Designed and/or selected ZFPs are modified according to the methods and compositions disclosed herein and may also be referred to as “engineered” ZFPs.

The term “naturally-occurring” is used to describe an object that can be found in nature, as distinct from being artificially produced by a human. For example, naturally occurring plant ZFPs are characterized by long spacers of diverse lengths between adjacent zinc finger components.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

With respect to fusion polypeptides, the term “operatively linked” can refer to the fact that each of the components performs the same function in linkage to the other component as it would if it were not so linked. For example, with respect to a fusion polypeptide in which a GMT-targeted ZFP DNA-binding domain is fused to a functional domain (or functional fragment thereof), the ZFP DNA-binding domain and the functional domain (or functional fragment thereof) are in operative linkage if, in the fusion polypeptide, the GMT-targeted ZFP DNA-binding domain portion is able to bind its target site and/or its binding site, while the functional domain (or functional fragment thereof) is able to modulate (e.g., activate or repress) transcription.

“Specific binding” between, for example, a ZFP and a specific target site means a binding affinity of at least 1×10⁶ M⁻¹.

A “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a GMT-targeted ZFP DNA-binding domain and a functional domain) and fusion nucleic acids (for example, a nucleic acid encoding the fusion polypeptides described herein). Examples of the second type of fusion molecule include, but are not limited to, a fusion between a triplex-forming nucleic acid and a polypeptide, and a fusion between a minor groove binder and a nucleic acid.

A “gene,” for the purposes of the present disclosure, includes a DNA region encoding a gene product (see below), as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Further, a promoter can be a normal cellular promoter or, for example, a promoter of an infecting microorganism such as, for example, a bacterium or a virus.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” and “augmentation of gene expression” refer to any process that results in an increase in production of a gene product. A gene product can be either RNA (including, but not limited to, mRNA, rRNA, tRNA, and structural RNA) or protein. Accordingly, gene activation includes those processes that increase transcription of a gene and/or translation of an mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes that increase translation include those that increase translational initiation, those that increase translational elongation and those that increase mRNA stability. In general, gene activation comprises any detectable increase in the production of a gene product, preferably an increase in production of a gene product by about 2-fold, more. preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more.

“Gene repression” and “inhibition of gene expression” refer to any process that results in a decrease in production of a gene product. A gene product can be either RNA (including, but not limited to, mRNA, rRNA, tRNA, and structural RNA) or protein. Accordingly, gene repression includes those processes that decrease transcription of a gene and/or translation of an mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes that decrease translation include those that decrease translational initiation, those that decrease translational elongation and those that decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integral value therebetween, more preferably between about 5- and about 10-fold or any integral value therebetween, more preferably between about 10- and about 20-fold or any integral value therebetween, still more preferably between about 20- and about 50-fold or any integral value therebetween, more preferably between about 50- and about 100-fold or any integral value therebetween, more preferably 100-fold or more. Most preferably, gene repression results in complete inhibition of gene expression, such that no gene product is detectable.

The term “modulate” refers to a change in the quantity, degree or extent of a function. For example, the GMT-targeted zinc finger-nucleotide binding polypeptides disclosed herein can modulate the activity of a promoter sequence by binding to a motif within the promoter, thereby inducing, enhancing or suppressing transcription of a gene operatively linked to the promoter sequence. Alternatively, modulation may include inhibition of transcription of a gene wherein the GMT-targeted zinc finger-nucleotide binding polypeptide binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript. Thus, “modulation” of gene expression includes both gene activation and gene repression.

Modulation can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP (see, e.g., Mistili & Spector, (1997) Nature Biotechnology 15:961-964); changes in signal transduction; changes in phosphorylation and dephosphorylation; changes in receptor-ligand interactions; changes in concentrations of second messengers such as, for example, cGMP, cAMP, 1P3, and Ca2⁺; changes in cell growth, changes in chemical composition (e.g., nutritional value), and/or changes in any functional effect of gene expression. Measurements can be made in vitro, in vivo, and/or ex vivo. Such finctional effects can be measured by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays; changes in intracellular second messengers such as cGMP and inositol triphosphate (IP₃); changes in intracellular calcium levels; cytokine release, and the like.

“Eucaryotic cells” include, but are not limited to, fungal cells (such as yeast), plant cells, animal cells, mammalian cells and human cells. Similarly, “prokaryotic cells” include, but are not limited to, bacteria.

A “regulatory domain” or “functional domain” refers to a protein or a polypeptide sequence that has transcriptional modulation activity, or that is capable of interacting with proteins and/or protein domains that have transcriptional modulation activity. Typically, a functional domain is covalently or non-covalently linked to a ZFP to modulate transcription of a gene of interest. Alternatively, a ZFP can act, in the absence of a functional domain, to modulate transcription. Furthermore, transcription of a gene of interest can be modulated by a ZFP linked to multiple functional domains.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one ore more amino acid or nucleotide substitutions. Methods for determining the function of a nucleic acid (e.g., coding function, ability to hybridize to another nucleic acid) are well known in the art. Similarly, methods for determining protein function are well known. For example, the DNA-binding function of a polypeptide can be determined, for example, by filter-binding, electrophoretic mobility-shift, or immunoprecipitation assays. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “target site” or “target sequence” is a sequence that is bound by a binding protein such as, for example, a ZFP. Target sequences can be nucleotide sequences (either DNA or RNA) or amino acid sequences. By way of example, a DNA target sequence for a three-finger ZFP is generally either 9 or 10 nucleotides in length, depending upon the presence and/or nature of cross-strand interactions between the ZFP and the target sequence. Target sequences can be found in any DNA or RNA sequence, including regulatory sequences, exons, introns, or any non-coding sequence.

A “target subsite” or “subsite” is the portion of a DNA target site that is bound by a single zinc finger, excluding cross-strand interactions. Thus, in the absence of cross-strand interactions, a subsite is generally three nucleotides in length. In cases in which a cross-strand interaction occurs (e.g., a “D-able subsite,” as described for example in co-owned PCT WO 00/42219, incorporated by reference in its entirety herein) a subsite is four nucleotides in length and overlaps with another 3- or 4-nucleotide subsite.

The term “effective amount” includes that amount which results in the desired result, for example, deactivation of a previously activated gene, activation of a previously repressed gene, or inhibition of transcription of a structural gene or translation of RNA.

As used herein, “genetically modified” or “transgenic” means a plant cell, plant part, plant tissue or plant which comprises one or more polynucleotide sequences which are introduced into the genome of a plant cell, plant part, plant tissue or plant by transformation or other suitable methods. The term “wild type” refers to an untransformed plant cell, plant part, plant tissue or plant, i.e., one where the genome does not include the selected polynucleotide sequences.

As used herein, “plant” refers to either a whole plant, a plant tissue, a plant part, such as pollen, seed or an embryo, a plant cell, or a group of plant cells. The class of plants that can be used is generally as broad as the class of seed-bearing higher plants amenable to transformation techniques, including both monocotyledonous and dicotyledonous plants. Seeds derived from plants regenerated from transformed plant cells, plant parts or plant tissues, or progeny derived from the regenerated transformed plants, may be used directly as feed or food, or can be altered by further processing. In the practice of the present disclosure, the most preferred plant seeds are those of Arabidopsis and Brassica. The transformation of the plants may be carried out in essentially any of the various ways known to those skilled in the art of plant molecular biology. These include, but are not limited to, microprojectile bombardment, microinjection, vacuum infiltration, electroporation of protoplasts or cells comprising partial cell walls, and Agrobacterium-mediated DNA transfer.

### Zinc Finger Proteins

Zinc finger proteins (ZFPs) are proteins that bind to DNA, RNA and/or protein, in a sequence-specific manner, by virtue of a metal stabilized domain known as a zinc finger. See, for example, Miller et al. (1985) EMBO J. 4:1609-1614; Rhodes et al. (1993) Sci. Amer. February:56-65; and Klug (1999) J. Mol. Biol. 293:215-218. There are at least 2 classes of ZFPs which co-ordinate zinc to form a compact DNA-binding domain. The first class includes the C₂H₂ ZFPs, that are composed of zinc fingers that contain two conserved cysteine residues and two conserved histidine residues in the following arrangement: -Cys-(X)₂₋₄-Cys-(X)₁₋₂-His-(X)₃₋₅-His (SEQ ID NO:2). C2H2 recognition regions are also referred to as “canonical.” A second class of ZFPs, referred to as Cys-Cys-His-Cys (SEQ ID NO: 3) (C₃H) ZFPs, have also been described, for example in Jiang et al. (1996) J. Biol. Chem. 271:10723-10730. C₃H ZFPS are a member of the family of non-canonical ZFPs, which include all non-C₂H₂ ZFPs. ZFPs including canonical, non-canonical and combinations of non-canonical and canonical zinc fingers can be utilized in the practice of the present disclosure.

Thus, zinc finger proteins are polypeptides that comprise zinc finger components. For example, zinc finger proteins can have one to thirty-seven fingers, commonly having 2, 3, 4, 5 or 6 fingers. Zinc finger DNA-binding proteins are described, for example, in Miller et al. (1985) EMBO J. 4:1609-1614; Rhodes et al. (1993) Scientific American February.:56-65; and Klug (1999) J. Mol. Biol. 293:215-218. A zinc finger protein recognizes and binds to a target site (sometimes referred to as a target sequence or target segment) that represents a relatively small portion of sequence within a target gene. Each component finger of a zinc finger protein typically binds to a subsite within the target site. The subsite includes a triplet of three contiguous bases on the same strand (sometimes referred to as the target strand). The three bases in the subsite can be individually denoted the 5′ base, the mid base, and the 3′ base of the triplet, respectively. The subsite may or may not also include a fourth base on the non-target strand that is the complement of the base immediately 3′ of the three contiguous bases on the target strand. The base immediately 3′ of the three contiguous bases on the target strand is sometimes referred to as the 3′ of the 3′ base. Alternatively, the four bases of the target strand in a four base subsite can be numbered 4, 3, 2, and 1, respectively, starting from the 5′ base.

The relative order of fingers in a zinc finger protein, from N-terminal to C-terminal, determines the relative order of triplets in the target sequence, in the 3′ to 5′ direction that will be recognized by the fingers. For example, if a zinc finger protein comprises, from Nterminal to C-terminal, first, second and third fingers that individually bind to the triplets 5′-GAC-3′, 5′-GTA-3′ and 5′-GGC-3′, respectively, then the zinc finger protein binds to the target sequence 5′-GGCGTAGAC-3′. If the zinc finger protein comprises the fingers in another order, for example, second finger, first finger, third finger, then the zinc finger protein binds to a target segment comprising a different permutation of triplets, in this example, 5′-GGCGACGTA-3′. See Berg et al. (1996) Science 271:1081-1086. The first amino acid of the alpha helical portion of the finger is assigned the number +1 and succeeding amino acids (proceeding toward the C-terminus) are assigned successively increasing numbers. The alpha helix generally extends to the residue following the second conserved histidine. The entire helix can therefore be of variable length, e.g., between 11 and 13 residues. The numbering convention used above is standard in the field for the region of a zinc finger conferring binding specificity, otherwise known as the recognition region.

### A. ZFPs Targeted to Plant GMT Genes

In general, GMT-targeted ZFPs are produced by first analyzing plant GMT sequences in order to select one or more target sites within GMT and engineer a ZFP that binds to these target site(s). GMT gene sequences can be readily obtained by methods described herein and include those sequences that are publicly available on any number of databases. Three-dimensional modeling for design of ZFPs can be used, but is not required.

In certain embodiments, the target site is present in an accessible region of cellular chromatin. Accessible regions can be determined as described in co-owned International Publications WO 01/83751 and WO 01/83732. If the target site is not present in an accessible region of cellular chromatin, one or more accessible regions can be generated as described in co-owned International Publication WO 01/83793. In additional embodiments, one or more GMT-targeted zinc finger binding components (or fusion molecules comprising these components) are capable of binding to cellular chromatin regardless of whether its target site is in an accessible region or not. For example, a ZFP as disclosed herein can be capable of binding to linker DNA and/or to nucleosomal DNA. Examples of this type of “pioneer” DNA binding domain are found in certain steroid receptors and in hepatocyte nuclear factor 3 (HNF3). Cordingley et al. (1987) Cell 48:261-270; Pina et al. (1990) Cell 60:719-731; and Cirillo et al. (1998) EMBO J. 17:244-254.

Exemplary methods for selecting target sites are described in WO 00/42219.

Once the target site(s) have been selected, the ZFPs are designed. Preferably, the ZFPs disclosed herein are composed wholly or partly of plant sequences, but have a non-plant structure. Methods of engineering such ZFPs are described, for example, in co-owned International Publications WO 02/057294 and WO 02/057293. As described in these documents, the non-plant structure of the GMT-targeted ZFP can be similar to that of any class of non-plant ZFP, for instance the C₂H₂ canonical class of ZFPs as exemplified by TFIIIA, Zif268 and Sp-1 or, a non-C2H2 structure, for example, a zinc finger protein in which one or more zinc coordinating fingers making up the zinc finger protein has any of the following sequences:

| | |
|---|---|
| X₃-B-X₂₋₄-Cys-X₁₂-His-X₁₋₇-His-X₄ | (SEQ ID NO:4) |
| X₃-Cys-X₂₋₄-B-X₁₂-His-X₁₋₇-His-X₄ | (SEQ ID NO:5) |
| X₃-Cys-X₂₋₄-Cys-X₁₂-Z-X₁₋₇-His-X₄ | (SEQ ID NO:6) |
| X₃-Cys-X₂₋₄-Cys-X₁₂-His-X₁₋₇-Z-X₄ | (SEQ ID NO:7) |
| X₃-B-X₂₋₄-B-X₁₂-His-X₁₋₇-His-X₄ | (SEQ ID NO:8) |
| X₃-B-X₂₋₄-Cys-X₁₂-Z-X₁₋₇-His-X₄ | (SEQ ID NO:9) |
| X₃-B-X₂₋₄-Cys-X₁₂-His-X₁₋₇-Z-X₄ | (SEQ ID NO:10) |
| X₃-Cys-X₂₋₄-B-X₁₂-Z-X₁₋₇-His-X₄ | (SEQ ID NO:11) |
| X₃-Cys-X₂₋₄-B-X₁₂-His-X₁₋₇-Z-X₄ | (SEQ ID NO:12) |
| X₃-Cys-X₂₋₄-Cys-X₁₂-Z-X₁₋₇-Z-X₄ | (SEQ ID NO:13) |
| X₃-Cys-X₂₋₄-B-X₁₂-Z-X₁₋₇-Z-X₄ | (SEQ ID NO:14) |
| X₃-B-X₂₋₄-Cys-X₁₂-Z-X₁₋₇-Z-X₄ | (SEQ ID NO:15) |
| X₃-B-X₂₋₄-B-X₁₂-His-X₁₋₇-Z-X₄ | (SEQ ID NO:16) |
| X₃-B-X₂₋₄-B-X₁₂-Z-X₁₋₇-His-X₄ | (SEQ ID NO:17) |
| X₃-B-X₂₋₄-B-X₁₂-Z-X₁₋₇-Z-X₄ | (SEQ ID NO:18) |

where X=any amino acid

B=any amino acid except cysteine

Z=any amino acid except histidine.

Furthermore, the ZFP can comprise sequences (e.g., recognition regions and/or backbones) from more than one class of ZFP. For example, a GMT-targeted ZFP can include a combination of canonical and non-canonical recognition regions inserted into a plant or other backbone. Selecting particular plant backbone residues to achieve the desired effector functions is disclosed herein and in co-owned International Publications WO 01/83751 and WO 01/83732. Fungal ZFPs can also be used as models for design and/or as sources of zinc finger sequences for GMT-targeted ZFPs. See, e.g., WO 96/32475. The documents cited herein also disclose methods of assessing binding affinity and/or specificity of ZFPs.

Sequences from any ZFP described herein can be altered by mutagenesis, substitution, insertion and/or deletion of one or more residues so that the non-recognition plant-derived residues do not correspond exactly to the zinc finger from which they are derived. Preferably, at least 75% of the GMT-targeted ZFP residues will correspond to those of the plant sequences, more often 90%, and most preferably greater than 95%.

Alterations in the recognition residues (i.e., positions −1 to +6 of the alpha helix) of any ZFP can be made so as to confer a desired binding specificity, for example as described in co-owned WO 00/42219; WO 00/41566; as well as U.S. Pat. Nos. 5,789,538; 6,007,408; 6,013,453; 6,140,081; 6,140,466; 6,242,568; as well as PCT publications WO 95/19431, WO 98/54311, WO 00/23464; WO 00/27878; WO 98/53057; WO 98/53058; WO 98/53059; and WO 98/53060.

Furthermore, in certain embodiments, ZFPs, as disclosed herein, contain additional modifications in their zinc fingers including, for example, non-canonical zinc fingers, in which a zinc-coordinating amino acid residue (i.e., cysteine and/or histidine) is substituted with a different amino acid. A GMT-targeted ZFP of this type can include any number of zinc finger components, and, in one embodiment, contains three zinc fingers. Any or all of the fingers can be a non-canonical finger(s). One or more of the component fingers of the protein can be naturally occurring zinc finger components, GMT-targeted plant components, canonical C₂H₂ fingers or combinations of these components.

As described in fuirther detail below, the GMT-targeted ZFPs described herein (and compositions comprising these ZFPs) can be provided to a plant or a plant cell as polypeptipdes or polynucleotides.

### B. Linkage

Two or more GMT-targeted zinc finger proteins can be linked to have a target site specificity that is, to a first approximation, the aggregate of that of the component zinc finger proteins. For example, a first GMT-targeted zinc finger protein having first, second and third component fingers that respectively bind to sequences represented by XXX, YYY and ZZZ can be linked to a second GMT-targeted zinc finger protein having first, second and third component fingers with binding specificities, AAA, BBB and CCC. The binding specificity of the combined first and second proteins is thus 5′-CCCBBBAAANZZZYYYXXX-3′, where N indicates a short intervening region (typically 0-5 bases of any type). In this situation, the target site can be viewed as comprising two target segments separated by an intervening segment.

Linkage of zinc fingers and zinc finger proteins can be accomplished using any of the following peptide linkers:

TGEKP (SEQ ID NO: 19) Liu et al. (1997) Proc. Natl. Acad. Sci. USA 94:5525-5530.

(G₄S)ₙ (SEQ ID NO: 20) Kim et al. (1996) Proc. Natl. Acad. Sci. USA 93:1156-1160.

GGRRGGGS (SEQ ID NO: 21)

LRQRDGERP (SEQ ID NO: 22)

LRQKDGGGSERP (SEQ ID NO: 23)

LRQKD(G₃S)₂ERP (SEQ ID NO: 24).

Alternatively, flexible linkers can be rationally designed using computer programs capable of modeling both DNA-binding sites and the peptides themselves, or by phage display methods. See, e.g., WO 99/45132 and WO 01/53480. In a further variation, non-covalent linkage can be achieved by fusing two zinc finger proteins with domains promoting heterodimer formation of the two zinc finger proteins. For example, one zinc finger protein can be fused withfos and the other withjun (see Barbas et al., WO 95/119431). Alternatively, dimerization interfaces can be obtained by selection. See, for example, Wang et al. (1999) Proc. Natl. Acad. Sci. USA 96:9568-9573.

### C. Fusion Molecules

The GMT-targeted zinc finger proteins described herein can also be used in the design of fusion molecules that facilitate regulation of GMT expression in plants. Thus, in certain embodiments, the compositions and methods disclosed herein involve fusions between at least one of the zinc finger proteins described herein (or functional fragments thereof) and one or more functional domains (or functional fragments thereof), or a polynucleotide encoding such a fusion. The presence of such a fusion molecule in a cell allows a functional domain to be brought into proximity with a sequence in a gene that is bound by the zinc finger portion of the fusion molecule. The transcriptional regulatory function of the functional domain is then able to act on the gene, by, for example, modulating expression of the gene.

In certain embodiments, fusion proteins comprising a GMT-targeted zinc finger DNA-binding domain and a functional domain are used for modulation of endogenous GMT expression. Modulation includes repression and activation of gene expression; the nature of the modulation generally depending on the type of functional domain present in the fusion protein. Any polypeptide sequence or domain capable of influencing gene expression (or functional fragment thereof) that can be fused to a DNA-binding domain, is suitable for use.

Suitable domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Seifpal et al., EMBO J. 11, 4961-4968 (1992)).

Additional exemplary activation domains include, but are not limited to, p300, CBP, PCAF,SRC1 PvALF, and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, Cl, AP1, ARF-5, -6, -7, and -8, CPRF1, CPRF4, MYC-RP/GP, and TRAB1.See, for example, Ogawa et al. (2000) Gene 245:21-29; Okanami et al. (1996) Genes Cells 1:87-99; Goffet al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gongetal. (1999)Plant Mol. Biol. 41:33-44; and Hobo et al. (1999) Proc. Natl. Acad. Sci. USA 96:15,348-15,353.

A preferred activation domain is the maize C1 activation domain. Goff et al. (1991) Genes & Devel 5:298-309.

An exemplary functional domain for fusing with a ZFP DNA-binding domain, to be used for repressing gene expression, is a KRAB repression domain from the human KOX-1 protein (see, e.g., Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994). Another suitable repression domain is methyl binding domain protein 2B (MBD-2B) (see, also Hendrich et al. (1999) Mamm Genome 10:906-912 for description of MBD proteins). Another useful repression domain is that associated with the v-ErbA protein. See, for example, Damm, et al. (1989) Nature 339:593-597; Evans (1989) Int. J. Cancer Suppl. 4:26-28; Pain et al. (1990) New Biol. 2:284-294; Sap et al. (1989) Nature 340:242-244; Zenke et al. (1988) Cell 52:107-119; and Zenke et al. (1990) Cell 61:1035-1049. Additional exemplary repression domains include, but are not limited to, thyroid hormone receptor (TR), SID, MBD1, MBD2, MBD3, MBD4, MBD-like proteins, members of the DNMT family (e.g., DNMT1, DNMT3A, DNMT3B), Rb, MeCP1 and MeCP2.See, for example, Zhang et al. (2000) Ann Rev Physiol 62:439-466; Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

Additional functional domains are disclosed, for example, in co-owned WO 00/41566. Further, insulator domains, chromatin remodeling proteins such as ISWI-containing domains and/or methyl binding domain proteins suitable for use in fusion molecules are described, for example, in co-owned International Publications WO 01/83793, PCT/US01/42377 and PCT/US01/44654.

In additional embodiments, targeted remodeling of chromatin, as disclosed, for example, in co-owned International Publication WO 01/83793, can be used to generate one or more sites in plant cell chromatin that are accessible to the binding of a functional domain/GMT-targeted ZFP fusion molecule.

Fusion molecules are constructed by methods of cloning and biochemical conjugation that are well known to those of skill in the art. Fusion molecules comprise a GMT-targeted ZFP binding domain and, for example, a transcriptional activation domain, a transcriptional repression domain, a component of a chromatin remodeling complex, an insulator domain or a functional fragment of any of these domains. In certain embodiments, fusion molecules comprise a GMT-targeted zinc finger protein and at least two functional domains (e.g., an insulator domain or a methyl binding protein domain and, additionally, a transcriptional activation or repression domain). Fusion molecules also optionally comprise a nuclear localization signal (such as, for example, that from the SV40 T-antigen or the maize Opaque-2 NLS) and an epitope tag (such as, for example, myc, his, FLAG or hemagglutinin). Fusion proteins (and nucleic acids encoding them) are designed such that the translational reading frame is preserved among the components of the fusion.

Methods of gene regulation using a functional domain, targeted to a specific sequence by virtue of a fused DNA binding domain, can achieve modulation of gene expression. Genes so modulated can be endogenous genes or exogenous genes. Modulation of gene expression can be in the form of activation (e.g., activating expression of GMT to increase levels of vitamin E in plant oils). As described herein, activation of GMT can be achieved by using a fusion molecule comprising a GMT-targeted zinc finger protein and a functional domain. The functional domain (e.g., insulator domain, activation domain, etc.) enables increased and/or sustained expression of the target gene. Alternatively, modulation can be in the form of repression. For any such applications, the fusion molecule(s) and/or nucleic acids encoding one or more fusion molecules can be formulated with an acceptable carrier, to facilitate introduction into and/or expression in plant cells, as is known to those of skill in the art.

### Polynucleotide and Polypeptide Delivery

The compositions described herein can be provided to the target cell in vitro or in vivo. In addition, the compositions can be provided as polypeptides, polynucleotides or combination thereof.

### A. Delivery of Polynucleotides

In certain embodiments, the compositions are provided as one or more polynucleotides. Further, as noted above, a GMT-targeted zinc finger protein-containing composition can be designed as a fusion between a zinc finger polypeptide and a functional domain that is encoded by a fusion nucleic acid. In both fusion and non-fusion cases, the nucleic acid can be cloned into intermediate vectors for transformation into prokaryotic or eukaryotic (e.g., plant) cells for replication and/or expression. Intermediate vectors for storage or manipulation of the nucleic acid or production of protein can be prokaryotic vectors, (e.g., plasmids), shuttle vectors, insect vectors, or viral vectors for example. A nucleic acid encoding a GMT-targeted zinc finger protein can also cloned into an expression vector, for administration to a bacterial cell, fungal cell, protozoal cell, plant cell, or animal cell, preferably a plant cell.

To obtain expression of a cloned nucleic acid, it is typically subdloned into an expression vector that contains a promoter to direct transcription. Suitable bacterial and eukaryotic promoters are well known in the art and described, e.g., in Sambrook et al., supra; Ausubel et al., supra; and Kriegler, Gene Transfer and Expression: A Laboratory Manual (1990). Bacterial expression systems are available in, e.g., E. coli, Bacillus sp., and Salmonella. Palva et al. (1983) Gene 22:229-235. Kits for such expression systems are commercially available. Eukaryotic expression systems for mammalian cells, yeast, and insect cells are well known in the art and are also commercially available, for example, from Invitrogen, Carlsbad, Calif. and Clontech, Palo Alto, Calif.

Plant expression vectors and reporter genes are also generally known in the art. (See, e.g., Gruber et al. (1993) in Methods of Plant Molecular Biology and Biotechnology, CRC Press.) Such systems include in vitro and in vivo recombinant DNA techniques, and any other synthetic or natural recombination. (See, e.g., Transgenic Plants: A Production System for Industrial and Pharmaceutical Proteins, Owen and Pen eds., John Wiliey & Sons, 1996; Transgenic Plants, Galun and Breiman eds, Imperial College Press, 1997; Applied Plant Biotechnology, Chopra, Malik, and Bhat eds., Science Publishers, Inc., 1999.)

The promoter used to direct expression of the nucleic acid of choice depends on the particular application. For example, a strong constitutive promoter is typically used for expression and purification. In contrast, when a protein is to be used in vivo, either a constitutive or an inducible promoter is used, depending on the particular use of the protein. In addition, a weak promoter can be used, when low but sustained levels of protein are required. The promoter typically can also include elements that are responsive to transactivation, e.g., hypoxia response elements and small molecule control systems such as tet-regulated systems and the RU-486 system. See, e.g., Gossen et al. (1992) Proc. Natl. Acad. Sci USA 89:5547-5551; Oligino etal.(1998) Gene Ther. 5:491-496; Wang et al. (1997) Gene Ther. 4:432-441; Neering et al. (1996) Blood 88:1147-1155; and Rendahl et al. (1998) Nat. Biotechnol. 16:757-761.

Promoters suitable for use in plant expression systems include, but are not limited to, viral promoters such as the 35S RNA and 19S RNA promoters of cauliflower mosaic virus (CaMV) (Brisson et al. (1984) Nature 310:511-514, Example 1); the coat protein promoter of TMV (Takamatsu et al. (1987) EMBO J. 6:307-311); plant promoters such as the small subunit of RUBISCO (Coruzzi et al. (1984) EMBO J. 3:1671-1680; Broglie et al. (1984) Science 224:838-843; plant heat shock promoters, e.g., soybean hsp17.5-E or hsp17.3-B (Gurley et al. (1986) Cell. Biol. 6:559-565) may be used. Other examples of promoters that may be used in expression vectors comprising nucleotides encoding GMT-targeted ZFPs include the promoter for the small subunit of ribulose-1,5-bis-phosphate carboxylase; promoters from tumor-inducing plasmids of Agrobacterium tumefaciens, such as the RUBISCO nopaline synthase (NOS) and octopine synthase promoters; bacterial T-DNA promoters such as mas and ocs promoters; or the figwort mosaic virus 35S promoter or others such as CaMV 19S (Lawton et al. (1987) Plant Molecular Biology 9:315-324), nos (Ebert et al. (1987) Proc. Natl. Acad. Sci. USA 84:5745-5749), Adh1 (Walker et al. (1987) Proc. Natl Acad. Sci. USA 84:6624-6628), sucrose synthase (Yang et al. (1990) Proc. Natl. Acad. Sci. USA 87:4144-4148), alpha-tubulin, ubiquitin, actin (Wang et al. (1992) Mol. Cell. Biol. 12:3399), cab (Sullivan et al. (1989) Mol. Gen. Genet. 215:431), PEPCase (Hudspeth et al. (1989) Plant Molecular Biology 12:579-589) or those associated with the R gene complex (Chandler et al. (1989) The Plant Cell 1:1175-1183). Further suitable promoters include the Z10 promoter from a gene encoding a 10 kD zein protein, a Z27 promoter from a gene encoding a 27 kD zein protein, inducible promoters, such as the light inducible promoter derived from the pea rbcS gene (Coruzzi et al. (1971) EMBO J. 3:1671) and the actin promoter from rice (McElroy et al. (1990) The Plant Cell 2:163 -171); seed specific promoters, such as the phaseolin promoter from beans, may also be used (Sengupta-Gopalan et al. (1985) Proc. Natl. Acad. Sci. USA. 83:3320-3324). Other suitable plant promoters are known to those of skill in the art.

Furthermore, additional promoters can be employed as described herein as many, if not all, genes have promoter regions capable of regulating gene expression. Additional promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide pairs. In addition to promoter sequences, enhancer sequences can also influence the level of gene expression. Some isolated promoter sequences can provide for gene expression of heterologous DNAs, that is a DNA different from the native or homologous DNA.

Thus, novel tissue-specific promoter sequences may be employed. cDNA clones from a particular tissue are isolated and those clones that are expressed specifically in that tissue are identified, for example, using Northern blotting. Preferably, the gene isolated is not present in a high copy number, but is relatively abundant in specific tissues. The promoter and control elements of corresponding genomic clones can then be localized using techniques well known to those of skill in the art.

In a preferred embodiment, the GMT-targeted ZFP polynucleotide sequence is under the control of the cauliflower mosaic virus (CaMV) 35S promoter. The caulimorvirus family has provided a number of exemplary promoters for transgene expression in plants, in particular, the (CaMV) 35S promoter. (See, e.g., Kay et al. (1987) Science 236:1299.) Additional promoters from this family such as the figwort mosaic virus promoter, the Commelina yellow mottle virus promoter, and the rice tungro bacilliform virus promoter have been described in the art, and may also be used in the methods and compositions disclosed herein. (See, e.g., Sanger et al. (1990) Plant Mol. Biol. 14:433-443; Medberry et al. (1992) Plant Cell 4:195-192; Yin and Beachy (1995) Plant J. 7:969-980.)

The promoters may be modified, if desired, to affect their control characteristics. For example, the CaMV 35S promoter may be ligated to the portion of the RUBISCO gene that represses the expression of RUBISCO in the absence of light, to create a promoter that is active in leaves, but not in roots. The resulting chimeric promoter may be used as described herein. Constitutive plant promoters such as actin and ubiquitin, having general expression properties known in the art may be used to express GMT-targeted ZFPs. (See, e.g., McElroy et al. (1990) Plant Cell 2:163-171; Christensen et al. (1992) Plant Mol. Biol. 18:675-689.)

Additionally, depending on the desired tissue, expression may be targeted to the endosperm, aleurone layer, embryo (or its parts as scutellum and cotyledons), pericarp, stem, leaves tubers, roots, etc. Examples of known tissue-specific promoters include the tuber-directed class I patatin promoter, the promoters associated with potato tuber ADPGPP genes, the soybean promoter of β-conglycinin (7S protein) which drives seed-directed transcription, and seed-directed promoters from the zein genes of maize endosperrn. (See, e.g., Bevan et al., 1986, Nucleic Acids Res. 14: 4625-38; Muller et al., 1990, Mol. Gen. Genet. 224: 136-46; Bray, 1987, Planta 172: 364-370 ; Pedersen et al., 1982, Cell 29: 1015-26.) Additional seed-specific promoters include the phaseolin and napin promoters.

In addition to a promoter, an expression vector typically contains a transcription unit or expression cassette that contains additional elements required for the expression of the nucleic acid in host cells, either prokaryotic or eukaryotic. A typical expression cassette thus contains a promoter operably linked, e.g., to the nucleic acid sequence, and signals required, e.g., for efficient polyadenylation of the transcript, transcriptional termination, ribosome binding, and/or translation termination.

The particular expression vector used to transport the genetic information into the cell is selected with regard to the intended use of the resulting ZFP polypeptide, e.g., expression in plants.

In addition, the recombinant constructs may include plant-expressible selectable or screenable marker genes for isolating, identifying or tracking of plant cells transformed by these constructs. Selectable markers include, but are not limited to, genes that confer antibiotic resistances (e.g., resistance to kanamycin or hygromycin) or herbicide resistance (e.g., resistance to sulfonylurea, phosphinothricin, or glyphosate). Screenable markers include, but are not limited to, the genes encoding beta-glucuronidase (Jefferson (1987) Plant Molec Biol. Rep 5:387-405), luciferase (Ow et al. (1986) Science 234:856-859), and the B and C1 gene products that regulate anthocyanin pigment production (Goff et al. (1990) EMBO J 9:2517-2522).

Thus, included within the terms selectable or screenable marker genes are also genes which encode a “secretable marker” whose secretion can be detected as a means of identifying or selecting for transformed cells. Examples include markers that encode a secretable antigen that can be identified by antibody interaction, or even secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of a gene that encodes a polypeptide that becomes sequestered in the cell wall, and which polypeptide includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). The use of the maize HPRG (Stiefel et al. (1990) The Plant Cell 2:785-793 1990) is preferred as this molecule is well characterized in terms of molecular biology, expression, and protein structure. However, any one of a variety of extensins and/or glycine-rich wall proteins (Keller et al. (1989) EMBO J. 8:1309-1314) could be modified by the addition of an antigenic site to create a screenable marker.

Possible selectable markers for use in connection with the present disclosure include, but are not limited to, a neo gene (Potrykus et al. (1985) Mol. Gen. Genet. 199:183-188) which codes for kanamycin resistance and can be selected for using kanamycin, G418, and the like; a bar gene which codes for bialaphos resistance; a gene which encodes an altered EPSP synthase protein (Hinchee et al. (1988) Bio/Technology 6:915-922) thus conferring glyphosate resistance; a nitrilase gene such as bxn from Klebsiella ozaenae which confers resistance to bromoxynil (Stalker et al. (1988) Science 242:419-423); a mutant acetolactate synthase gene (ALS) which confers resistance to imidazolinone, sulfonylurea or other ALS-inhibiting chemicals (European Patent Application 154,204, 1985); a DHFR gene which confers methotrexate resistance (Thillet et al. (1988) J Biol. Chem. 263:12500-12508); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571, 1987).

Illustrative embodiments of selectable marker genes capable of being used in systems to select transformants are genes that encode the enzyme phosphinothricin acetyltransferase, such as the bar gene from Streptomyces hygroscopicus or the pat gene from Streptomyces viridochromogenes (U.S. Pat. No. 5,550,318, which is incorporated by reference herein). The enzyme phosphinothricin acetyl transferase (PAT) inactivates the active ingredient in the herbicide bialaphos, phosphinothricin (PPT). PPT inhibits glutamine synthetase, (Murakami et al. (1986) Mol Gen. Genet. 205:42-50; Twell et al., (1989) Plant Physiol. 91:1270-1274) causing rapid accumulation of ammonia and cell death. The success in using this selective system in conjunction with monocots was particularly surprising because of the major difficulties that have been reported in transformation of cereals (Potrykus (1989) Trends Biotech. 7:269-273).

Screenable markers that may be employed include, but are not limited to, a beta-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al. (1988) In: Chromosome Structure and Function: Impact of New Concepts, 18th Stadler Genetics Symposium, Jp.P. Gustafson and R. Appels, eds. (New York: Plenum Press) pp. 263-282); a beta-lactamase gene (Sutcliffe (1978) Proc. Natl. Acad. Sci. USA 75:3737-3741), which encodes an enzyme for which various chromogenic substrates are known (e.g., PADAC, a chromogenic cephalosporin); a xylE gene (Zukowsky et al. (1983) Proc. Natl. Acad. Sci. USA. 80:1101) which encodes a catechol dioxygenase that can convert chromogenic catechols; an alpha-amylase gene (Ikuta et al. (1990) Bio/technology 8:241-242); a tyrosinase gene (Katz et al. (1983) J. Gen. Microbiol. 129 (Pt. 9) 2703-2714) which encodes an enzyme capable of oxidizing tyrosine to DOPA and dopaquinone, which in turn condenses to form the easily detectable compound melanin; a beta-galactosidase gene, which encodes an enzyme for which there are chromogenic substrates; a luciferase (lux) gene (Ow et al. (1986) Science 234:856-859), which allows for bioluminescence detection; or an aequorin gene (Prasher et al. (1985) Biochem. Biophys. Res. Comm. 126:1259-1268), which may be employed in calcium-sensitive bioluminescence detection, or a green fluorescent protein gene (Niedz et al (1995) Plant Cell Reports 14:403).

Genes from the maize R gene complex are contemplated to be particularly useful as screenable markers. The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. A gene from the R gene complex is useful for maize transformation, because the expression of this gene in transformed cells does not harm the cells. Thus, an R gene introduced into such cells will cause the expression of a red pigment and, if stably incorporated, can be visually scored as a red sector. If a maize line carries dominant alleles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together. Anthocyanin pigments can be used as markers in plants other than maize. See, for example, Lloyd et al. (1992) Science 258:1773-1775. Hence, alleles of the maize R gene and the genes involved in maize anthhocyanin biosynthesis are useful in a wide variety of plants.

R gene regulatory regions may be employed in chimeric constructs in order to provide mechanisms for controlling the expression of chimeric genes. More diversity of phenotypic expression is known at the R locus than at any other locus (Coe et al. (1988) in Corn and Corn Improvement, eds. Sprague, G. F. & Dudley, J. W. (Am. Soc. Agron., Madison, Wis.), pp. 81-258). Regulatory regions obtained from regions 5′ to the structural R gene can be used in directing the expression of genes, e.g., insect resistance, drought resistance, herbicide tolerance or other protein coding regions. For the purposes of the present disclosure, it is believed that any of the various R gene family members may be successfully employed (e.g., P, S, Lc, etc.). However, the most preferred will generally be Sn (particularly Sn:bo13). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. This system can be developed for populational screening for bioluminescence, such as on tissue culture plates, or even for whole plant screening.

Elements of the present disclosure are exemplified in detail through the use of particular marker genes. However in light of this disclosure, numerous other possible selectable and/or screenable marker genes will be apparent to those of skill in the art in addition to the oned set forth herein. Therefore, it will be understood that the foregoing discussion is exemplary rather than exhaustive. In light of the techniques disclosed herein and the general recombinant techniques known in the art, it is possible to introduce any gene, including those encoding marker genes and/or GMT-targeted ZFPs, into a recipient cell to generate a transformed plant cell, e.g., a dicot or a monocot cell.

Other elements that are optionally included in expression vectors also include a replicon that functions in E. coli (or in the prokaryotic host, if other than E. coli), a selective marker that functions in a prokaryotic host, e.g., a gene encoding antibiotic resistance, to permit selection of bacteria that harbor recombinant plasmids, and unique restriction sites in nonessential regions of the vector to allow insertion of recombinant sequences.

Standard transfection methods can be used to produce bacterial, mammalian, yeast, insect, other cell lines or, preferably, plants that express large quantities of GMT-targeted zinc finger proteins, which can be purified, if desired, using standard techniques. See, e.g., Colley et al. (1989) J. Biol. Chem. 264:17619-17622; and Guide to Protein Purification, in Methods in Enzymology, vol. 182 (Deutscher, ed.) 1990. Transformation of non-plant eukaryotic cells and prokaryotic cells are performed according to standard techniques. See, e.g., Morrison (1977) J. Bacteriol. 132:349-351; Clark-Curtiss et al. (1983) in Methods in Enzymology 101:347-362 (Wu et al., eds), Sambrook, supra and Ausubel, supra.

Transformation systems for plants are also known. (See, e.g., Weissbach & Weissbach, Methods for Plant Molecular Biology, Academic Press, NY, Section VIII, pp. 421-463 (1988); Grierson & Corey, Plant Molecular Biology, 2d Ed., Blackie, London, Ch. 7-9 (1988)). For example, Agrobacterium is often successfully employed to introduce nucleic acids into plants. Such transformation preferably uses binary Agrobacterium T-DNA vectors which can be used to transform dicotyledonous plants, monocotyledonous plants and plant cells (Bevan (1984) Nuc. Acid Res. 12:8711-8721; Horsch et al. (1985) Science 227:1229-1231; Bevan et al. (1982) Ann. Rev. Genet 16:357-384; Rogers et al. (1986) Methods Enzymol. 118:627-641; Hemalsteen et al. (1984) EMBO J 3:3039-3041). In embodiments that utilize the Agrobacterium system for transforming plants, the recombinant DNA constructs typically comprise at least the right T-DNA border sequence flanking the DNA sequences to be transformed into the plant cell. In preferred embodiments, the sequences to be transferred are flanked by the right and left T-DNA border sequences. The design and construction of such T-DNA based transformation vectors are well known to those skilled in the art.

Other gene transfer and transformation methods include, but are not limited to, protoplast transformation through calcium-, polyethylene glycol (PEG)- or electroporation-mediated uptake of naked DNA (see Paszkowski et al. (1984) EMBO J 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276); electroporation of plant tissues (D'Halluin et al. (1992) Plant Cell 4:1495-1505); microinjection, silicon carbide mediated DNA uptake (Kaeppler et al. (1990) Plant Cell Reporter 9:415-418), microprojectile bombardment (see Klein et al. (1983) Proc. Nat. Acad. Sci. USA 85:4305-4309; and Gordon-Kamm et al. (1990) Plant Cell 2:603-618); direct gene transfer, in vitro protoplast transformation, plant virus-mediated transformation, liposome-mediated transformation, vacuum infiltration (Bechtold et al. (1998) Methods Mol. Biol. 82:259-266 (1998); Clough et al. (1998) Plant J, 16(6):735-743; and Ye et al. (1999) Plant J. 19(3): 249-257) and ballistic particle acceleration (See, e.g., Paszkowski et al. (1984) EMBO J. 3:2717-2722; U.S. Pat. Nos. 4,684,611; 4,407,956; 4,536,475; Crossway et al., (1986) Biotechniques 4:320-334; Riggs et al (1986) Proc. Natl. Acad. Sci USA 83:5602-5606; Hinchee et al. (1988) Biotechnology 6:915-921; U.S. Pat. No. 4,945,050.)

A wide variety of host cells, plants and plant cell systems can be used, including, but not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yarn), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, canola) and plants used for experimental purposes (e.g., Arabidopsis).

GMT-targeted ZFPs and the resulting gene product the ZFP modulates (GMT and downstream products such as tocopherol) can also be produced from seed by way of seed-based production techniques using, for example, canola (rape seed), corn, soybeans, rice and barley seed, and the GMT-targeted ZFP, and/or sequences encoding it, can be recovered during seed germination. See, e.g., PCT Publication Numbers WO 99/40210; WO 99/16890; WO 99/07206; U.S. Pat. No.: 5,866,121; and U.S. Pat. No.: 5,792,933; and all references cited therein.

### B. Delivery of Polypeptides

In additional embodiments, GMT-targeted ZFPs or fusion proteins comprising GMT-targeted ZFPs are administered directly to target plant cells. In certain in vitro situations, the target cells are cultured in a medium containing a fusion protein comprising one or more functional domains fused to one or more of the GMT-targeted ZFPs described herein. An important factor in the administration of polypeptide compounds in plants is ensuring that the polypeptide has the ability to traverse a cell wall. However, proteins, viruses, toxins, ballistic methods and the like have the ability to translocate polypeptides across a plant cell wall.

For example, “plasmodesmata” is the term given to the structures involved in cellto-cell transport of endogenous and viral proteins and ribonucleoprotein complexes (RNPCs) in plants. Examples of viruses which can be linked to a GMT-targeted plant zinc finger polypeptide (or fusion containing the same) for facilitating its uptake into plant cells include tobacco mosaic virus (Oparka et al. (1997) Plant J. 12:781-789); rice phloem thioredoxin (Ishiwatari et al. (1998) Planta 205:12-22); potato virus X (Cruz et al. (1998) Plant Cell 10:495-510) and the like. Other suitable chemical moieties that provide enhanced cellular uptake can also be linked, either covalently or non-covalently, to the ZFPs. Toxin molecules also have the ability to transport polypeptides across cell walls.

Particle-mediated delivery techniques (e.g., ballistic injection) as described above regarding nucleic acids can also be used to introduce polypeptides into a plant cell.

### Production and Characterization of Stable Transgenic Plants

Techniques for generating transgenic plants are known in the art (see, e.g., Swain W F (1991) TIBTECH9: 107-109; Ma J. K C et al. (1994) Eur J Immunology 24:131-138; Hiatt A et al. (1992) FEBS Letters 307:71-75; Hein M B et al. (1991) Biotechnology Progress 7: 455-461; Duering K (1990) Plant Molecular Biology 15: 281-294). Non-limiting examples of transformation procedures are described herein and include agrobacterium-mediated transformation, microinjection, particle bombardment, and vacuum infiltration.

Typically, after effecting delivery of a polynucleotide to recipient plant cells by any of the methods discussed above, successfully transformed cells are identified for further culturing and plant regeneration. As mentioned above, in order to improve the ability to identify transformants, one may desire to employ a selectable or screenable marker gene as, or in addition to, the expressible sequence. In this case, one would then generally assay the potentially transformed cell population by exposing the cells to a selective agent or agents, or one would screen the cells for the desired marker gene trait.

### A. Selection

An exemplary embodiment of methods for identifying successfully transformed cells involves exposing the recipient cultures to a selective agent, such as a metabolic inhibitor, an antibiotic, herbicide or the like. Cells that have been transformed and have stably integrated a marker gene conferring resistance to the selective agent used, will grow and divide in culture. Sensitive cells will not be amenable to further culturing.

To use the bar-bialaphos or the EPSPS-glyphosate selective system, tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate will typically be preferred, it is proposed that ranges of at least about 0.1-50 mg/l bialaphos or at least about 0.1-50 mM glyphosate will find utility in the practice of the present disclosure. Tissue can be placed on any porous, inert, solid or semi-solid support, including but not limited to filters and solid culture medium. Bialaphos and glyphosate are provided as non-limiting examples of agents suitable for selection of transformants.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants. In a similar fashion, the introduction of the C1 and B genes will result in pigmented cells and/or tissues.

The enzyme luciferase is also useful as a screenable marker in the context of the present disclosure. In the presence of the substrate luciferin, cells expressing luciferase emit light that can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells that are expressing luciferase and manipulate those in real time.

It is further contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective growth inhibition of transformants and nontransformants alike, thus causing the selection technique to be ineffective. Selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. Combinations of selection and screening will enable one to identify transformants in a wider variety of cell and tissue types.

### B. Regeneration and Seed Production

Cells that survive the exposure to the selective agent, or cells that have been scored positive in a screening assay, may be cultured in media that supports regeneration of plants, for example dicamba or 2,4-D, NAA, NAA+2,4-D and/or picloram. Tissue is preferably maintained on a basic medium with growth regulators (optionally agar) until sufficient tissue is available to begin plant regeneration efforts, or following repeated rounds of manual selection, until the morphology of the tissue is suitable for regeneration then transferred to medium conducive to maturation of embryoids. Shoot development typically signals the time to transfer to medium lacking growth regulator.

The transformed cells, identified by selection or screening and cultured in an appropriate medium that supports regeneration, will then be allowed to mature into plants. After the regenerating plants have reached the stage of shoot and root development, they may be transferred to a greenhouse for further growth and testing.

Mature plants are then obtained from cell lines that are known to express the GMT-targeted ZFP. If possible, the regenerated plants are self-pollinated. In addition, pollen obtained from the regenerated plants is crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants in a process known as backcross conversion. When a sufficient number of crosses to the recurrent inbred parent have been completed in order to produce a product of the backcross conversion process that is substantially isogenic with the recurrent inbred parent except for the presence of the introduced polynucleotide sequence(s), the plant is self-pollinated at least once in order to produce a homozygous backcross converted inbred. Progeny of these plants are true breeding and the weight percentage of vitamin E in a plant part, e.g., the seeds, or the amount of starch in these progeny are compared to the weight percentage of vitamin E in the recurrent parent inbred, in the field under a range of environmental conditions. Methods of determining weight percentages are well known in the art.

Alternatively, seed from transformed monocot or dicot plants regenerated from transformed tissue cultures is grown in the field and self-pollinated to generate true breeding plants.

Seed from the fertile transgenic plants can then be evaluated for the presence and/or expression of GMT and/or vitamin E. A substantial activation of the production of GMT is an increase in the activity of GMT per cell and/or the weight percent of GMT and/or vitamin E, preferably at least 2-fold, more preferably at least 5-fold, even more preferably at least 20-fold and even more preferably at least 100-fold or more, as compared the levels normally present in a non-transformed plant.

Once a transgenic plant (e.g., seed) expressing the GMT-targeted ZFP sequence and having an increase in GMT expression is obtained, seeds can be used to develop true breeding plants. The true breeding plants are used to develop a line of plants exhibiting increased expression of GMT.

### C. Determination of Stably Transformed Plant Tissues

To confirm the presence of a ZFP as described herein within the regenerating plants, or seeds or progeny derived from the regenerated plant, a variety of assays may be performed. Such assays include, for example, molecular biological assays well known to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and hence it will be necessary to prepare RNA for analysis from these tissues. PCR techniques can be used for detection and quantitation of RNA produced from introduced polynucleotide(s). For example, GMT mRNA can be detected by real-time PCR, e.g., TaqMan® analysis. In the use of PCR for analysis of RNA, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by RNA (“Northern”) blotting. This technique will demonstrate the presence of an RNA species and give information about the integrity of that RNA. The presence or absence of an RNA species can also be determined using dot or slot blot hybridizations. These techniques are modifications of Northern blotting and will only demonstrate the presence or absence of an RNA species.

Expression of GMT itself may also be evaluated by specifically identifying GMT, vitamin E, or by evaluating the phenotypic changes brought about by their expression. Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Very frequently the expression of a gene product is determined by evaluating the phenotypic results of its expression. These assays also may take many forms including but not limited to analyzing changes in the chemical composition, morphology, or physiological properties of the plant.

Thus, the GMT-targeted ZFP (or fusion polypeptides comprising the GMT-targeted ZFPs described herein) can then be used to modulate GMT (and vitamin E) expression in plant cells using GMT-targeted ZFPs to optimize vitamin E content of the crop. A GMT-targeted ZFP can be targeted to a region outside of the GMT coding region of the gene of interest and, in certain embodiments, is targeted to a region outside of known regulatory region(s) of the gene. In these embodiments, additional molecules, exogenous and/or endogenous, can optionally be used to facilitate repression or activation of gene expression. The additional molecules can also be fusion molecules, for example, fusions between a ZFP and a functional domain such as an activation or repression domain. See, for example, co-owned WO 00/41566.

All references cited herein are hereby incorporated by reference in their entirety for all purposes.

The following examples are presented as illustrative of, but not limiting, the claimed subject matter.

### EXAMPLES

### Example 1

### Production of Modified Plant Zinc Finger Binding Proteins

This example describes a strategy to select amino acid sequences for plant zinc finger backbones from among existing plant zinc finger sequences, and subsequent conceptual modification of the selected plant zinc finger amino acid sequences to optimize their DNA binding ability. Oligonucleotides used in the preparation of polynucleotides encoding proteins containing these zinc fingers in tandem array are then described.

### A. Selection of Plant Zinc Finger Backbones

A search was conducted for plant zinc fingers whose backbone sequences (i.e., the portion of the zinc finger outside of the −1 through +6 portion of the recognition helix) resembled that of the SP-1 consensus sequence described by Berg (1992) Proc. Natl. Acad. Sci. USA 89:11,109-11,110. The sequences selected included the two conserved cysteine residues, a conserved basic residue (lysine or arginine) located two residues to the C-termninal side of the second (i.e. C-terminal) cysteine, a conserved phenylalanine residue located two residues to the C-terminal side of the basic residue, the two conserved histidine residues, and a conserved arginine residue located two residues to the C-terninal side of the first (i.e., N-terminal) conserved histidine. The amino acid sequences of these selected plant zinc finger backbones (compared to the SP-1 consensus sequence) are shown below, with conserved residues shown in bold and X referring to residues located at positions −1 through +6 in the recognition helix (which will differ among different proteins depending upon the target sequence):

| SP-1 consensus: | |
|---|---|
| YKCPECGKSFSXXXXXXXHQRTHTGEKP | (SEQ ID NO:25) |
| F1: | |
| KKKSKGHECPICFRVFKXXXXXXXHKRSHTGEKP | (SEQ ID NO:26) |
| F2 | |
| YKCTVCGKSFSXXXXXXXHKRLHTGEKP | (SEQ ID NO:27) |
| F3 | |
| FSCNYCQRKFYXXXXXXXHVRIH | (SEQ ID NO:28) |
| −5 −1 5 | |

The first finger (F1) was chosen because it contained a basic sequence N-terminal to the finger that is also found adjacent to the first finger of SP-1. The finger denoted F1 is a Petunia sequence, the F2 and F3 fingers are Arabidopsis sequences.

### B. Modification of Plant Zinc Finger Backbones

Two of the three plant zinc fingers (F1 and F3, above) were modified so that their amino acid sequences more closely resembled the sequence of SP-1, as follows. (Note that the sequence of SP-1 is different from the sequence denoted “SP-1 consensus.”) In F3, the Y residue at position −2 was converted to a G, and the sequence QNKK (SEQ ID NO:29) was added to the C-terminus of F3. The QNKK sequence is present C-terminal to the third finger of SP-1, and permits greater flexibility of that finger, compared to fingers 1 and 2, which are flanked by the helix-capping sequence T G E K/R K/P (SEQ ID NO:30). Such flexibility can be beneficial when the third finger is modified to contain a non-C₂H₂ structure. Finally, several amino acids were removed from the N-terminus of F1. The resulting zinc finger backbones had the following sequences:

| | |
|---|---|
| KSKGHECPICFRVFKXXXXXXXHKRSHTGEKP | (SEQ ID NO:31) |
| YKCTVCGKSFSXXXXXXXHKRLHTGEKP | (SEQ ID NO:32) |
| FSCNYCQRKFGXXXXXXXHVRIHQNKK | (SEQ ID NO:33) |

Amino acid residues denoted by X, present in the recognition portion of these zinc fingers, are designed or selected depending upon the desired target site, according to methods disclosed, for example, in co-owned WO 00/41566 and WO 00/42219, and/or references cited supra.

### C. Nucleic Acid Sequences Encoding Backbones for Modified Plant ZFPs

The following polynucleotide sequences are used for design of a three-finger plant ZFP that contains the F1, F2 and F3 backbones described above. Polynucleotides encoding multi-finger ZFPs are designed according to an overlapping oligonucleotide method as described in, for example, co-owned WO 00/41566 and WO 00/42219. Oligonucleotides H1, H2 and H3 (below) comprise sequences corresponding to the reverse complement of the recognition helices of fingers 1-3 respectively; accordingly, nucleotides denoted by N will vary depending upon the desired amino acid sequences of the recognition helices, which, in turn, will depend upon the nucleotide sequence of the target site. Oligonucleotides PB1, PB2 and PB3 encode the betasheet portions of the zinc fingers, which are common to all constructs. Codons used frequently in Arabidopsis and E. coli were selected for use in these oligonucleotides.

| | |
|---|---|
| H1: | |
| 5′-CTC ACC GGT GTG AGA ACG CTT GTG NNN NNN NNN NNN NNN NNN NNN CTT GAA AAC ACG GAA-3′ | (SEQ ID NO:34) |
| H2: | |
| 5′-TTC ACC AGT ATG AAG ACG CTT ATG NNN NNN NNN NNN NNN NNN NNN AGA AAA AGA CTT ACC-3′ | (SEQ ID NO:35) |
| H3: | |
| 5′-CTT CTT GTT CTG GTG GAT ACG CAC GTG NNN NNN NNN NNN NNN NNN NNN ACC GAA CTT ACG | (SEQ ID NO:36) |
| CTG-3′ | |
| PB1: | |
| 5′-AAGTCTAAGGGTCACGAGTGCCCAATCTGCTTCCGTGTTTTCAAG-3′ | (SEQ ID NO:37) |
| PB2: | |
| 5′-TCTCACACCGGTGAGAAGCCATACAAGTGCACTGTTTGTGGTAAGTCTTTTTCT-3′ | (SEQ ID NO:38) |
| PB3: | |
| 5′-CTTCATACTGGTGAAAAGCCATTCTCTTGCAACTACTGCCAGCGTAAGTTCGGT-3′ | (SEQ ID NO:39) |

Briefly, these six oligonucleotides were annealed and amplified by polymerase chain reaction. The initial amplification product was reamplified using primers that were complementary to the initial amplification product and that also contained 5′ extensions containing restriction enzyme recognition sites, to facilitate cloning. The second amplification product was inserted into a vector containing, for example, one or more functional domains, nuclear localization sequences, and/or epitope tags. See, for example, co-owned WO 00/41566 and WO 00/42219.

### Example 2

### Construction of Vectors for Expression of Modified Plant ZFPs

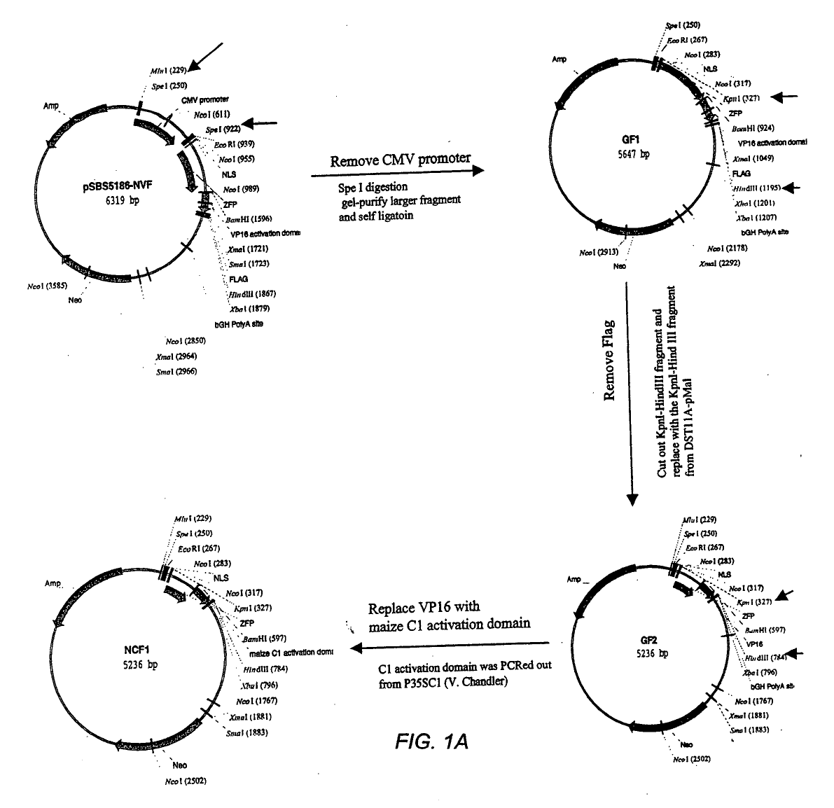

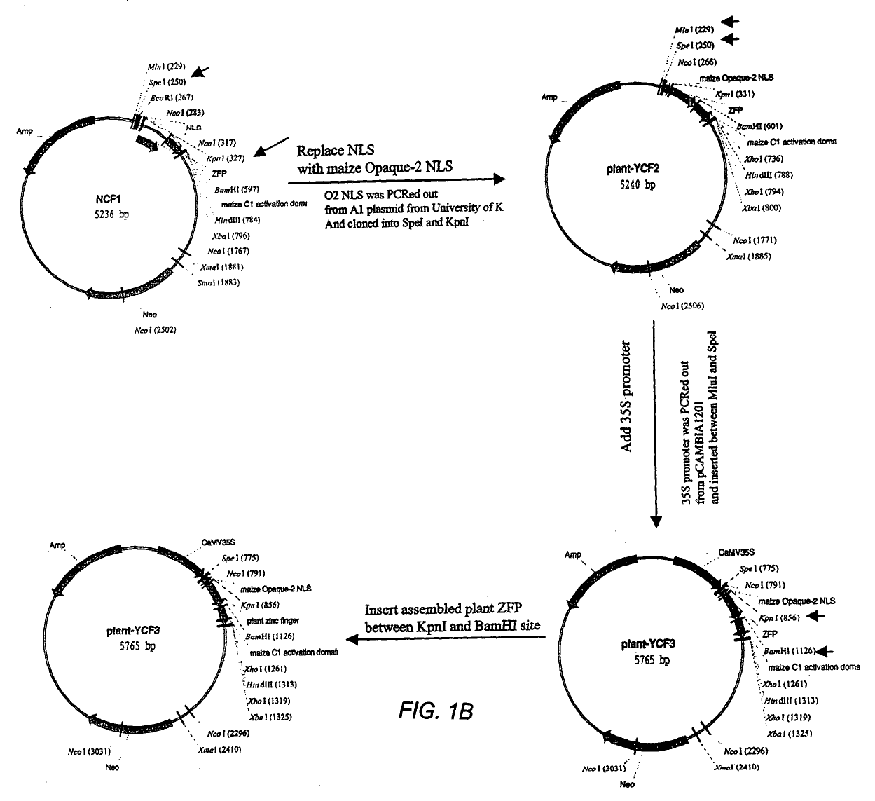

YCF3 was generated as shown schematically in FIG. 1. The starting construct was a plasmid containing a CMV promoter, a SV40 nuclear localization sequence (NLS), a ZFP DNA binding domain, a Herpesvirus VP16 transcriptional activation domain and a FLAG epitope tag (pSB5186-NVF). This construct was digested with Spel to remove the CMV promoter. The larger fragment was gel-purified and self-ligated to make a plasmid termed GF1. GF1 was then digested with KpnI and HindIII, releasing sequences encoding the ZFP domain, the VP16 activation domain, and the FLAG epitope tag, then the larger fragment was ligated to a KpnI/HindIII fragment containing sequences encoding a ZFP binding domain and a VP16 activation domain, named GF2. This resulted in deletion of sequences encoding the FLAG tag from the construct.

GF2 was digested with BaniHI and Hindlll, releasing a small fragment encoding the VP16 activation domain, and the larger fragment was purified and ligated to a BamHL/HindIII digested PCR fragment containing the maize C1 activation domain (Goff et al. (1990) EMBO J. 9:2517-2522) (KpnI and HindIII sites were introduced into the PCR fragment through KpnI and HindIII site-containing primers) to generate NCFI. A PCR fragment containing a Maize Opaque-2 NLS was digested with SpeI/KpnI and ligated to the larger fragment from KpnI/SpeI digested NCF1 to produce YCF2. YCF2 was then digested with MluI and SpeI and the larger fragment was ligated to an MluI and SpeI digested PCR fragment containing the plant-derived CaMV 35S promoter (MluI and Spel sites were introduced into the PCR fragment through MluI or SpeI site containing primers) to generate the YCF3 vector.

Sequences encoding GMT-targeted ZFP binding domains can be inserted, as KpnI/BamHI fragments, into KpnI/BamHI-digested YCF3 to generate constructs encoding ZFP-functional domain fusion proteins for modulation of gene expression in plant cells. For example, a series of Arabidopsis and Brassica GMT-ZFP domains, described in Examples 3 and 5 below, were inserted into KpnI/Bam.HI-digested YCF3 to generate expression vectors encoding GMT-ZFP-activation domain fusion polypeptides that enhance expression of plant (e.g., Arabidopsis thaliana, Brassica) GMT.

### Example 3

### Modified Plant ZFP Designs for Regulation of an Arabidopsis thaliana Gamma Tocopherol Methyltransferase (GMT) Gene

GMT-targeted zinc finger proteins were designed to recognize various target sequences in the Arabidopsis GMT gene (GenBank Accession Number AAD38271. Table 1 shows the nucleotide sequences of the various GMT target sites, and the amino acid sequences of zinc fingers that recognize the target sites. Sequences encoding these binding domains were prepared as described in Example 1 and inserted into YCF3 as described in Example 2.

TABLE 1

| ZFP # | Target | F1 | F2 | F3 |
|---|---|---|---|---|
| 1 | GTGGACGAGT | RSDNLAR | DRSNLTR | RSDALTR |
| | (SEQ ID NO:40) | (SEQ ID NO:41) | (SEQ ID NO:42) | (SEQ ID NO:43) |
| 2 | CGGGATGGGT | RSDHLAR | TSGNLVR | RSDHLRE |
| | (SEQ ID NO:44) | (SEQ ID NO:45) | (SEQ ID NO:46) | (SEQ ID MO:47) |
| 3 | TGGTGGGTGT | RSDALTR | RSDHLTT | RSDHLTT |
| | (SEQ ID NO:48) | (SEQ ID NO:49) | (SEQ ID NO:50) | (SEQ ID NO:51) |
| 4 | GAAGAGGATT | QSSNLAR | RSDNLAR | QSGNLTR |
| | (SEQ ID NO:52) | (SEQ ID NO:53) | (SEQ ID NO:54) | (SEQ ID NO:55) |
| 5 | GAGGAAGGGG | RSDHLAR | QSGNLAR | RSDNLTR |
| | (SEQ ID NO:56) | (SEQ ID NO:57) | (SEQ ID NO:58) | (SEQ ID NO:59) |
| 6 | TGGGTAGTC | ERGTLAR | QSGSLTR | RSDNLTT |
| | (SEQ ID NO:60) | (SEQ ID NO:61) | (SEQ ID NO:62) | (SEQ ID NO:63) |
| 7 | GGGGAAAGGG | RSDHLTQ | QSGNLAR | RSDHLSR |
| | (SEQ ID NO:64) | (SEQ ID NO:65) | (SEQ ID NO:66) | (SEQ ID NO:67) |
| 8 | GAAGAGGGTG | QSSHLAR | RSDNLAR | QSGNLAR |
| | (SEQ ID NO:68) | (SEQ ID NO:69) | (SEQ ID NO:70) | (SEQ ID NO:71) |
| 9 | GAGGAGGATG | QSSNLQR | RSDNALR | RSDNLQR |
| | (SEQ ID NO:72) | (SEQ ID NO:73) | (SEQ ID NO:74) | (SEQ ID NO:75) |
| 10 | GAGGAGGAGG | RSDNALR | RSDNLAR | RSDNLTR |
| | (SEQ ID NO:76) | (SEQ ID NO:77) | (SEQ ID NO:78) | (SEQ ID NO:79) |
| 11 | GTGGCGGCTG | QSSDLRR | RSDELQR | RSDALTR |
| | (SEQ ID NO:80) | (SEQ ID NO:81) | (SEQ ID NO:82) | (SEQ ID NO:83) |
| 12 | TGGGGAGAT | QSSNLLAR | QSGHLQR | RSDHLTT |
| | (SEQ ID NO:84) | (SEQ ID NO:85) | (SEQ ID NO:86) | (SEQ ID NO:87) |
| 13 | GAGGAAGCT | QSSDLRR | QSGNLAR | RSDNLTR |
| | (SEQ ID NO:88) | (SEQ ID NO:89) | (SEQ ID NO:90) | (SEQ ID NO:91) |
| 14 | GCTTGTGGCT | DRSHLTR | TSGHLTT | QSSDLTR |
| | (SEQ ID NO:92) | (SEQ ID NO:93) | (SEQ ID NO:94) | (SEQ ID NO:95) |
| 15 | GTAGTGGATG | QSSNLAR | RSDALSR | QSGSLTR |
| | (SEQ ID NO:96) | (SEQ ID NO:97) | (SEQ ID NO:98) | (SEQ ID NO:99) |
| 16 | GTGTGGGATT | QSSNLAR | RSDHLTT | RSDALTR |
| | (SEQ ID NO:100) | (SEQ ID NO:101) | (SEQ ID NO:102) | (SEQ ID NO:103) |

### Example 4

### Modulation of Expression of an Arabidopsis thaliana Gamma Tocopherol Methyltransferase (GMT) Gene

Arabidopsis thaliana protoplasts were prepared and transfected with plasmids encoding GMT-targeted ZFP-activation domain fusion polypeptides. Preparation of protoplasts and polyethylene glycol-mediated transfection were performed as described. Abel et al. (1994) Plant Journal 5:421-427. The different plasmids contained the GMT-targeted ZFP binding domains described in Table 1, inserted as KpnI/BamHI fragrnents into YCF3.

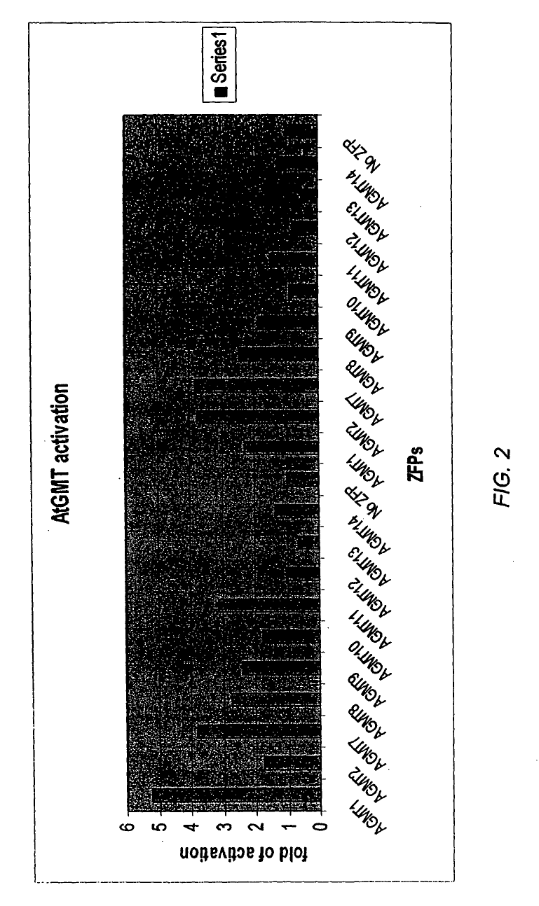

At 18 hours after transfection, RNA was isolated from transfected protoplasts, using an RNA extraction kit from Qiagen (Valencia, Calif.) according to the manufacturer's instructions. The RNA was then treated with DNase (RNase-free), and analyzed for GMT mRNA content by real-time PCR (TaqMan®). Table 2 shows the sequences of the primers and probe used for TaqMan® analysis. Results for GMT mRNA levels were normalized to levels of 18S rRNA. These normalized results are shown in FIG. 2 as fold-activation of GMT mRNA levels, compared to protoplasts transfected with carrier DNA (denoted “No ZFP” in FIG. 2). The results indicate that expression of the GMT gene was enhanced in protoplasts that were transfected with plasmids encoding fusions between a transcriptional activation domain and a GMT-targeted ZFP binding domain targeted to the GMT gene.

TABLE 2

| | SEQUENCE | |
|---|---|---|
| GMT forward | 5′-AATGATCTCGCGGCTGCT-3′ | (SEQ ID NO:104) |
| primer | | |
| GMT reverse primer | 5′-GAATGGCTGATCCAACGCAT-3′ | (SEQ ID NO:105) |
| GMT probe | 5′-TCACTCGCTCATAAGGCTTCCTTCCAAGT-3′ | (SEQ ID NO:106) |
| 18S forward primer | 5′-TGCAACAAACCCCGACTTATG-3′ | (SEQ ID NO:107) |
| 18S reverse primer | 5′-CCCGCGTCGACCTTTTATC-3′ | (SEQ ID NO:108) |
| 18S probe | 5′-AATAAATGCGTCCCTT-3′ | (SEQ ID NO:109) |

### Example 5

### Modified Plant ZFP Designs for Regulation of a Brassica napus Gamma Tocopherol Methyltransferase (GMT) Gene

GMT-targeted plant zinc finger proteins were designed to recognize various target sequences in the Brassica napus GMT gene. The Brassica GMT sequence is disclosed in WO 02/063022. Target sites within the Brassica sequence were selected, and zinc fingers were designed to bind the selected target sites as described, for example, in co-owned U.S. Pat. No. 6,453,242. Table 3 shows the nucleotide sequences of the various GMT target sites, and the amino acid sequences of zinc fingers that recognize the target sites. Sequences encoding these binding domains were prepared as described in Example 1 and inserted into YCF3 as described in Example 2.

TABLE 3

| ZFP # | Target | F1 | F2 | F3 |
|---|---|---|---|---|
| C3 | GATGCTGGT | QSSHLAR | QSSDLTR | TSGNLTR |
| | | (SEQ ID | (SEQ ID | (SEQ ID |
| | | NO:110) | NO:111) | NO:112) |
| C4 | GAGGAAGAT | QSSNLAR | QSGNLAR | RSDNLTR |
| | | (SEQ ID | (SEQ ID | (SEQ ID |
| | | NO:113) | NO:114) | NO:115) |
| C5 | GAAGAAGAG | RSDNLAR | QSGNLAR | QSGNLAR |
| | | (SEQ ID | (SEQ ID | (SEQ ID |
| | | NO:116) | NO:117) | NO:118) |
| C6 | GAGGTTGGA | QSGHLAR | TSGALTR | RSDNLTR |
| | | (SEQ ID | (SEQ ID | (SEQ ID |
| | | NO:119) | NO:120) | NO:121) |
| C7 | GATGATGAT | QSSNLR | TSGNLTR | TSGNLTR |
| | | (SEQ ID | (SEQ ID | (SEQ ID |
| | | NO:122) | NO:123) | NO:124) |
| C8 | CGGGGAGAG | RSSNLAR | QSGHLQR | RSDHLRE |
| | | (SEQ ID | (SEQ ID | (SEQ ID |
| | | NO:125) | NO:126) | NO:127) |
| C9 | TAGTTGGAA | QSGNLAR | RSDALTT | RSDNLTT |
| | | (SEQ ID | (SEQ ID | (SEQ ID |
| | | NO:128) | NO:129) | NO:130) |
| C10 | GTAGAGGAC | DRSNLTR | RSDNLAR | QSGSLTR |
| | | (SEQ ID | (SEQ ID | (SEQ ID |
| | | NO:131) | NO:132) | NO:133) |
| C12 | GAGGTTGGC | DRSHLTR | TSGALTR | RSDNLTR |
| | | (SEQ ID | (SEQ ID | (SEQ ID |
| | | NO:134) | NO:135) | NO:136) |

### Example 6

### Modulation of Expression of a Brassica napus Gamma Tocopherol Methyltransferase (GMT) Gene

Brassica nabus protoplasts were prepared and transfected with plasmids encoding GMT-targeted ZFP-activation domain fision polypeptides essentially as described in Example 4 except that mannitol concentration was 0.55M (instead of 0.4M) and the concentration of protoplasts before transfection was 0.2×10⁶ (instead of 1×10⁶). The different plasmids contained the GMT-targeted ZFP binding domains described in Table 3, inserted as KpnI/BamHI fragments into YCF3.

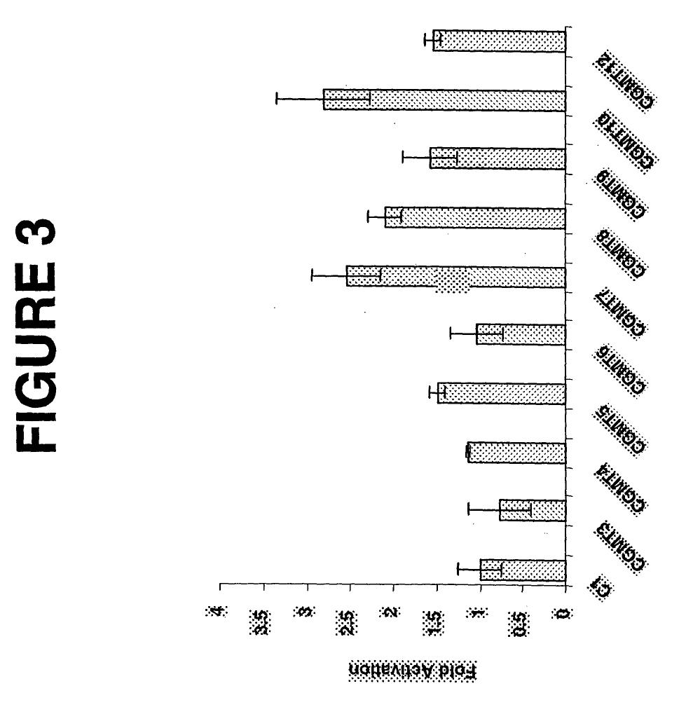

At 18 hours after transfection, RNA was isolated from transfected protoplasts, using an RNA extraction kit from Qiagen (Valencia, Calif.) according to the manufacturer's instructions. The RNA was then treated with DNase (RNase-free), and analyzed for GMT mRNA content by real-time PCR (TaqMan®). Table 4 shows the sequences of the primers and probe used for TaqMan® analysis. Results for GMT mRNA levels were normalized to levels of GAPDH mRNA. These normalized results are shown in FIG. 3 as fold-activation of GMT mRNA levels, compared to protoplasts transfected with DNA encoding an activation domain only (denoted “C1” in FIG. 3). The results indicate that expression of the GMT gene was enhanced in canola protoplasts that were transfected with plasmids encoding fusions between a transcriptional activation domain and a GMT-targeted ZFP binding domain.

TABLE 4

| | SEQUENCE | |
|---|---|---|
| cGMT forward primer* | 5′-CAATGGAAAGCGGTGAGCATAT-3′ | (SEQ ID NO:137) |
| cGMT reverse primer | 5′-TCCTTCCTCCTGGAGCCG-3′ | (SEQ ID NO:138) |
| cGMT probe | 5′-CTGACAAGGCCAAGTTCGTGAAGGAATTG-3′ | (SEQ ID NO:139) |
| GAPDH forward primer | 5′-GATCATCAAGATTGTATCTGATC-3′ | (SEQ ID NO:140) |
| GAPDH reverse primer | 5′-CGGTTCCTTCGATAACTAAGTC-3′ | (SEQ ID NO:141) |
| GAPDH probe | 5′-CGGTTCCTTCGATAACTAAGTC-3′ | (SEQ ID NO:142) |

*“c” refers to canola

### Example 7

### Transgenic Arabidopsis

Transgenic Arabidopsis plants were prepared as follows.

### A. Agrobacterium Preparation

Agrobacterium strain GV3101 was streaked on AB plates (1×AB salts (per liter, 1 g NH₄Cl; 300 mg MgSO₄; 150 mgKCl; 10 mg CaCl₂; 2.5 mg FeSO₄)+1×AB buffer (per liter, 3 g K₂HPO₄; 1.1 5 g NaH₂PO₄; pH to 7.2)+0.2% glucose +15 g agar) and incubated at 30° C. for 2 days. A single colony was picked and used to inoculate 2 mL of liquid MG-LB medium (per liter, 10 g tryptone; 5 g yeast extract; 10 g mannitol; 1.9 g L-glutamic acid; 0.5 g KH₂PO₄; 0.2 g NaCl; 0.2 g MgSO₄ 7H₂O, pH to 7.2). This culture was incubated overnight with shaking at 30° C.

The next morning, the 2 mL culture was used to inoculate 100 mL of liquid MG-LB medium and grown for 4 to 6 hours with shaking at 30° C. The culture was chilled on ice, transferred to a sterile centrifuge bottle and centrifuged at 4000×G at 4° C. for 5 minutes. The bacterial pellet was resuspended in 1 mL ALB medium (per liter, 10 g tryptone; 5 g yeast extract). 100 μL aliquots of the resuspended culture were placed into chilled 1.5 mL tubes and flash frozen in liquid nitrogen. The tubes were thawed on ice and 3 μL of the AGMT-ZFP plasmid DNA (AGMT5, AGMT6, AGMT7, AGMT8, AGMT 9 and AGMT 10) was mixed gently with the cells. The tubes were again flash frozen in liquid nitrogen and then allowed to thaw and incubate at 37° C. for 5 minutes. The cultures were then transferred to 2 mL of MG-LB medium and incubated with shaking for 3 hours at 30° C. After incubation, the cultures were pelleted by centrifugation, resuspend in 1 mL of 1×AB salts, and plated on AB minimal plates supplemented with 100 μg/mL kanamycin. The plates were incubated for 2 days at 30°. Single colonies were selected for transformation of Arabidopsis thaliana plants.

### B. Transformation of Arabidopsis thaliana

Transformation was conducted essentially as described in Clough SJ and Bent AF (1998) “Floral dip: a simplified method for Agrobacterium-mediated transformation of Arabidopsis thaliana” Plant J. 16:735-43. (See, also Bechtold, N., Ellis, J., and Pelletier, G. (1993) “In planta Agrobacterium-mediated gene transfer by infiltration of adult Arabidopsis thaliana plants” C. R. Acad. Sci. Paris, Life Sciences 316:1194-1199 and http://plantpath.wisc.edu/˜afb/protocol.html, 6-11-2001). Wild type Arabidopsis seedlings, Columbia ecotype, were grown under long days (16 hours light, 8 hours dark) at 22° C. in pots of Ready Earth soil less mixture covered with window screen material. When the plants were approximately 4-6 weeks old, the primary flowering bolts were removed and the secondary bolts were allowed to emerge and grow until they were up to 10 cm long.

A single colony of transformed Agrobacterium tumefaciens containing each of the AGMT-ZFPs (see above) was grown in 2 mL of YEP (per liter, 10 g peptone; 10 g yeast extract; 5 g NaCl) plus 100 μg/mL kanamycin at 30° C. overnight. This 2 mL culture was used to inoculate a 500 mL culture of YEP plus 100 μg/mL kanamycin and again grown at 30° C. overnight. The resulting cultures were centrifuged at 5000×G at 4° C. for 15 minutes and the bacterial pellets were resuspended in 5% sucrose to an OD₆₀₀ of approximately 0.8. 0.05% Silwet L-77 (Sentre Chemical Company, Memphis, Tenn.) was added to the culture after resuspension. The plants were then dipped with a gentle agitation in the Agrobacterium solution for about 90 seconds. The pots were then placed in a tray under a plastic wrap cover to maintain high humidity for 16 to 24 hours. The plastic wrap was removed the next day and the plants were allowed to grow, mature and set seed. T0 seeds were collected and subjected to bialaphos selection.

### C. Selection of Primary AGMT-ZFP Transformnants of Arabidopsis thaliana

Each AGMT-ZFP vector contains the Bar gene which confers resistance to the herbicide bialaphos to use as a selection marker for transformation. Thus, T1 plants containing the AGMT-ZFPs were selected by resistance to the herbicide. (See, also, Kobayashi et al. (1995) Jpn J Genet 70(3):409-422).

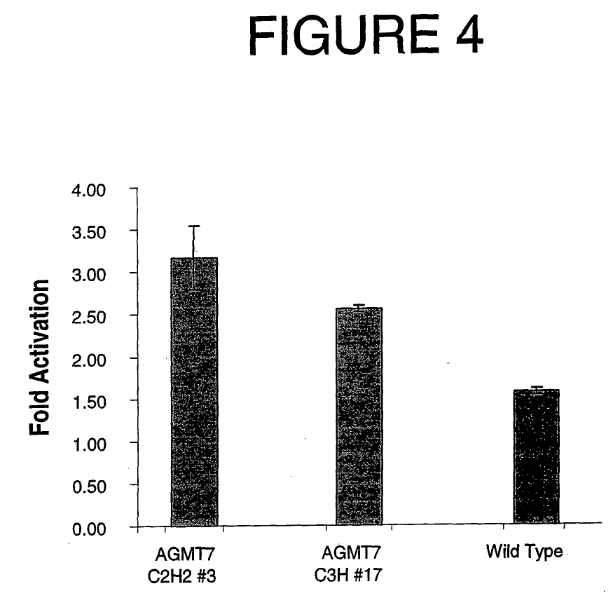

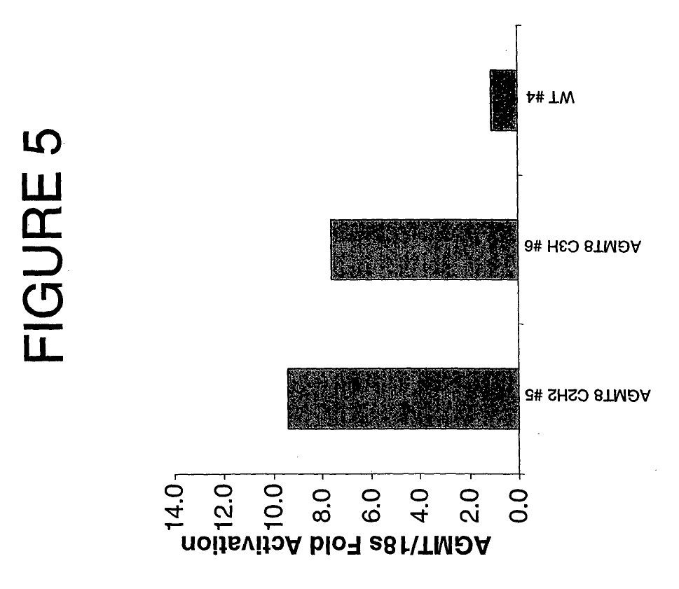

T0 seeds were sprinkled on top of Ready Earth soil less mixture in 4″ plastic pots and watered via subirrigation. The pots were then placed at 4° C. for vernalization. After 48 hours, the pots were removed from the cold and the seedlings were allowed to germinate and grow under longs days (16 hours light, 8 hours dark) at 22° C. After one week, the seedlings were sprayed until wet with a solution of 100 μg bialaphos plus 0.005% Silwet L-77. The plants were sprayed again 2 days later. After an additional week of growth, the T1 seedlings were apparent among the non-transformed plants as they grew green and healthy. The T1 seedlings were transferred to individual pots and allowed to grow until seed set. FIGS. 4 and 5 show RNA analysis from these plants and demonstrate that GMT-targeted ZFPs can be used to create transgenic plants that overexpress GMT.

Although the foregoing methods and compositions have been described in detail for purposes of clarity of understanding, certain modifications, as known to those of skill in the art, can be practiced within the scope of the appended claims. All publications and patent documents cited herein are hereby incorporated by reference in their entirety for all purposes to the same extent as if each were so individually denoted.

What is claimed is:

1. An engineered zinc finger protein that binds to a target site in a plant gamma-tocopherol methyl transferase (GMT) gene.

2. The zinc finger protein of claim 1, wherein said protein modulates expression of GMT.

3. A fusion polypeptide comprising a zinc finger protein according to claim 1 and at least one functional domain.

4. The fusion polypeptide of claim 3, wherein the functional domain is an activation domain.

5. An isolated polynucleotide encoding the zinc finger protein of claim 1.

6. An expression vector comprising the isolated polynucleotide of claim 5.

7. The expression vector of claim 6, comprising a plant promoter.

8. The expression vector of claim 7, wherein the promoter is tissue specific.
